(19) United States
(12) Patent Application Publication
(10) Pub. No.: US 2025/0282830 A1
(43) Pub. Date: Sep. 11, 2025

(54) ANTI-VIRAL PEPTIDE COMPOSITIONS AND METHODS TO IMPROVE BIOLOGICAL ACTIVITY THEREOF

(71) Applicant: NANYANG TECHNOLOGICAL UNIVERSITY, Singapore (SG)

(72) Inventor: Nam-Joon CHO, Singapore (SG)

(21) Appl. No.: 18/286,816

(22) PCT Filed: Apr. 13, 2022
(86) PCT No.: PCT/SG2022/050213

§ 371 Date: Oct. 13, 2023

(30) Foreign Application Priority Data
Apr. 13, 2021 (SG) 10202103747S

(51) Int. Cl. C07K 14/005; A61K 38/00; A61P 31/14

(52) U.S. Cl. CPC C07K 14/005; A61P 31/14; A61K 38/00; C12N 2770/24222; C12N 2770/24232; C12N 2770/24271

(57) ABSTRACT

Disclosed herein are peptides of 27 amino acid residues, and compositions comprising said peptides. Also disclosed herein are their uses in treating or preventing a viral infection.

### FIELD OF INVENTION

Provided herein are anti-infective peptides and uses thereof. Such anti-infective peptides are useful against bacteria and viruses. Also provided herein are compositions comprising said anti-infective peptides.

### BACKGROUND

The listing or discussion of a prior-published document in this specification should not necessarily be taken as an acknowledgement that the document is part of the state of the art or is common general knowledge.

In recent years, several promising classes of inhibitors have been identified that target viral membranes by exploiting the differences between virus particles and mammalian cells, such as membrane repair capacity and geometrical dimensions. In the course of studying the CLR01 molecule (that was originally designed as a “molecular tweezer” to bind lysine and arginine residues to block HIV-amyloid complex formation), it was found that micromolar concentrations of CLR01 also destabilize the lipid envelope surrounding HIV, hepatitis C, and herpes simplex virus particles. It was later shown that CLR01 is active against Zika and Ebola viruses as well. Notably, CLR01 exhibits inhibitory activity in human seminal fluid (as it was originally aimed at inhibiting HIV transmission in semen) but not in serum and displays low toxicity in vitro and in vivo. These findings have led it to be considered as a topical microbicide candidate. The antiviral selectivity of CLR01 is speculated to arise from the compositional differences between host cell membranes and virion membranes (the latter often has a greater predominance of raft-like components due to the virus budding site) as well as the membrane repair capacity of mammalian cells but not viruses.

Other classes of small molecules have been discovered that work as photosensitizing agents and cause oxidation of unsaturated phospholipids in viral membranes via light-dependent generation of singlet oxygen species. Such alterations can decrease membrane fluidity and affect membrane ordering to hinder cell-virus fusion. For example, LJ001 is an aryl methyldiene rhodanine derivative that exhibits potent antiviral activity against Rift Valley fever, yellow fever, and West Nile viruses at a nanomolar concentration range. Biophysical experiments indicated that LJ001 treatment affects membrane organization without causing permeation. To enable in vivo applications, improved versions of LJ001 were designed that had greater potency, red-shifted absorption spectra, increased quantum yields, and higher bioavailability. Such lead compounds, including JL122, were able to delay death in a lethal mouse model of Rift Valley fever virus infection.

Rigid amphipathic fusion inhibitors (RAFIs) are another class of small molecules that act against enveloped viruses and are believed to target viral membranes by inhibiting the formation of negative membrane curvature required for host cell fusion along with causing light-induced lipid oxidation. As exemplified by aUY11, RAFIs exhibit nanomolar antiviral activity in vitro. Structure-activity relationship studies have revealed that different parts of the RAFI molecular structure are responsible for antiviral and cytotoxic activities. Hence, it is possible to design RAFIs with potent antiviral activity and low cytotoxicity, although available studies are limited to in vitro testing.

Aside from small molecule-based antivirals, peptide-based antivirals, such as the 18-mer C5A peptide (derived from the N-terminal region of hepatitis C virus NS5A protein), have been reported to inhibit a wide range of enveloped viruses in vitro. The C5A is a promising antiviral peptide that has been tested as a topical microbicide to prevent vaginal HIV transmission ex vivo in humanized mice and in nonhuman primates. Most efforts were focused on progressing membrane-disruptive compounds toward topical microbicide applications due to the challenges of translating their in vitro antiviral activities to in vivo animal models.

Therefore, there is a need to seek new antipathogenic compositions with improved biological activity for medical applications.

### SUMMARY OF INVENTION

Aspects and embodiments of the invention will now be discussed by reference to the following numbered clauses.

1. A peptide of 27 amino acid residues in length comprising the following amino acid sequence: X₁X₂X₃WLX₄X₅X₆WX₇WX₈X₉X₁₀X₁₁X₁₂X₁₃DFX₁₄X₁₅X₁₆LX₁₇X₁₈KX₁₉, wherein:
X₁ is S, G, D, or A;
X₂ is G, D, E, or S;
X₃ is S, D, N, or T;
X₄ is R, H, Y, or W;
X₅ is D, I, T, or E;
X₆ is I or V;
X₇ is D, E, or N;
X₈ is I or V;
X₉ is C or L;
X₁₀ is E, T, S, I, or H;
X₁₁ is I or V;
X₁₂ is L or V;
X₁₃ is S, T, or A;
X₁₄ is K or R;
X₁₅ is T, N, A, V or L;
X₁₆ is W or C;
X₁₇ is K, Q, T, S, or G;
X₁₈ is A or S; and
X₁₉ is L, I, or F (SEQ ID NO: 12)
and wherein:
each amino acid is independently an L- or D-amino acid;
the functional groups of the N- and C-termini of the peptide are:
   (A) unchanged from the standard N- and C-termini functional groups;
   (B) reversed, such that the N-terminus has a carboxylic acid functional group and the C-terminus has an amino functional group;
   (C) the N-terminus and C-terminus both have an amino functional group; or
   (D) the N-terminus and C-terminus both have a carboxylic acid functional group, provided that, when the peptide has the sequence: SGSWLRDVWDWICTVLTDFKTWLQSKL (SEQ ID NO: 2), the functional groups of the N- and C-termini of the peptide are selected from (B) to (D) above only.

2. The peptide according to Clause 1, wherein the peptide comprises the following amino acid sequence: X₁X₂X₃WLRX₅X₆WDWX₈CX₁₀X₁₁X₁₂X₁₃DFKX₁₅WLX₁₇X₁₈KX₁₉

wherein:
X₁ is S or G;
X₂ is G or D;
X₃ is S, D or T;
X₅ is D or I;
X₆ is I or V;
X₈ is I or V;
X₁₀ is T or S;
X₁₁ is I or V;
X₁₂ is L or V;
X₁₃ is S, T or A;
X₁₅ is T, N or A;
X₁₇ is K or S;
X₁₈ is A or S; and
X₁₉ is L or I (SEQ ID NO: 13)
   and wherein each amino acid is independently an L- or D-amino acid.

3. The peptide according to Clause 1 or Clause 2, wherein the peptide comprises the following amino acid sequence: SX₂X₃WLRDX₆WDWVCX₁₀VLX₁₃DFKX₁₅WLX₁₇X₁₈KX₁₉ wherein:
X₂ is G or D;
X₃ is S, D or T;
X₆ is I or V;
X₁₀ is T or S;
X₁₃ is S or T;
X₁₅ is T, N or A;
X₁₇ is K or S;
X₁₈ is A or S; and
X₁₉ is L or I (SEQ ID NO: 14)
   and wherein each amino acid is independently an L- or D-amino acid.

4. The peptide according to any one of the preceding clauses, wherein the peptide comprises the following amino acid sequence:

SGX₃WLRDIWDWVCX₁₀VLX₁₃DFKTWLSAKX₁₉

wherein:
X₃ is S, D or T;
X₁₀ is T or S;
X₁₃ is S or T; and
X₁₉ is L or I (SEQ ID NO: 15)
   and wherein each amino acid is independently an L- or D-amino acid.

5. The peptide according to Clause 1, wherein the peptide has a peptide sequence from the following list:

(i)
(SEQ ID NO: 1)
SGSWLRDIWDWICEVLSDFKTWLKAKL;
(ii)
(SEQ ID NO: 2)
SGSWLRDVWDWICTVLTDFKTWLQSKL;
(iii)
(SEQ ID NO: 3)
SGSWLRDVWDWVCTILTDFKNWLTSKL;
(iv)
(SEQ ID NO: 4)
SGSWLRDIWEWVCSILTDFKNWLSAKL;
(v)
(SEQ ID NO: 5)
SDDWLRIIWDWVCSVVSDFKAWLSAKI;
(vi)
(SEQ ID NO: 6)
SGDWLRIIWDWVCSVVSDFKTWLSAKI;
(vii)
(SEQ ID NO: 7)
SDDWLRTIWDWVCSVLADFKAWLSAKI;
(viii)
(SEQ ID NO: 8)
GDDWLHDIWDWVCIVLSDFKTWLSAKI;
(ix)
(SEQ ID NO: 9)
DGNWLYDIWNWVCTVLADFKLWLGAKI;
(x)
(SEQ ID NO: 10)
AESWLWEVWDWVLHVLSDFKTCLKAKF;
and
(xi)
(SEQ ID NO: 11)
GSTWLRDIWDWVCTVLSDFRVWLKSKL,

and wherein each amino acid is independently an L- or D-amino acid.

6. The peptide according to any one of the preceding clauses, wherein each amino acid in each sequence is an L-amino acid.

7. The peptide according to any one of Clauses 1 to 5, wherein each amino acid in each sequence is a D-amino acid.

8. The peptide according to any one of Clauses 1 to 5, wherein the amino acids in each sequence are a mixture of L- and D-amino acids.

9. The peptide according to any one of the preceding clauses, wherein the functional groups of the N- and C-termini of the peptide are:
(AI) the N-terminus and C-terminus both have an amino functional group; or
(AII) the N-terminus and C-terminus both have a carboxylic acid functional group.

10. A pegylated peptide comprising a peptide of 27 amino acid residues in length as described in any one of Clauses 1 to 9 linked to one, two or more polyethylene glycol (PEG) polymers, optionally wherein:
(aa) the one, two or more PEG polymers is linked to the N-terminus and/or C-terminus of the peptide; and/or
(ab) each of the one, two or more PEG polymers has a molecular weight range of from 500 to 5,000 Daltons;
(ac) each of the one, two or more PEG polymers is branched or unbranched.

11. A pharmaceutical composition comprising a peptide according to any one of Clauses 1 to 9 or a pegylated peptide according to Clause 10, and a pharmaceutically acceptable carrier.

12. The pharmaceutical composition according to Clause 11, wherein the composition further comprises one or more pharmaceutically acceptable excipients and adjuvants.

13. The pharmaceutical composition according to Clause 11 or Clause 12, wherein the composition is formulated for subcutaneous, intravenous or intraperitoneal administration.

14. A vial containing a pharmaceutical composition according to any one of Clauses 11 to 13, optionally wherein the pharmaceutical composition is lyophilized.

15. A peptide according to any one of Clauses 1 to 9, or a pegylated peptide according to Claim 10 for use in medicine.

16. A method of treating or preventing a viral infection in a subject, the method comprising administering a pharmaceutically effective amount of a peptide according to any one of Clauses 1 to 9, or a pegylated peptide according to Claim 10.

17. A peptide according to any one of Clauses 1 to 9, or a pegylated peptide according to Claim 10 for use in treating or preventing a viral infection.

18. Use of a peptide according to any one of Clauses 1 to 9, or a pegylated peptide according to Claim 10 in the preparation of a medicament for treating or preventing a viral infection.

19. The method according to Clause 16, the peptide or pegylated peptide for use according to Clause 17, or the use according to Clause 18, wherein the viral infection is selected from one or more of the group consisting of a virus of the coronaviridae or, more particularly, flaviviridae, togaviridae, filoviridae, arenaviridae, poxviridae, bunyaviridae, and retroviridae families.

20. The method, peptide or pegylated peptide for use, or use according to Clause 19, wherein the viral infection is a mosquito-borne virus, optionally wherein the viral infection is selected from one or more of the group consisting of dengue, Zika, yellow fever, West Nile, and Chikungunya.

21. The method, peptide or pegylated peptide for use, or use according to Clause 19, wherein the viral infection is selected from one or more of the group consisting of HCV, HDV, JEV, COVID19, SARS, MERS-CoV, MERS, SARS-CoV-2, alphacoronavirus, betacoronavirus, gammacoronavirus or, more particularly, dengue, Chikungunya virus, Ebola, HIV, West Nile, Zika, yellow fever, and influenza.

### DRAWINGS

FIG. 1 depicts the helix net wheel diagrams.

FIG. 2 depicts the circular dichroism (CD) spectra of peptides in water.

FIG. 3 depicts the CD spectra of peptides in 50% 2,2,2-trifluoroethanol (TFE).

FIG. 4 depicts the quartz crystal microbalance-dissipation (QCM-D) frequency shift (top) and energy dissipation shift (bottom) data for tracking peptide-induced vesicle rupture.

FIG. 5 depicts the QCM-D frequency shift (top) and energy dissipation shift (bottom) data for tracking peptide-induced supported lipid bilayer interactions.

FIG. 6 depicts the time-lapse fluorescence microscopy of peptide-induced vesicle rupture at different peptide concentrations and rupture percentage summary.

FIG. 7 depicts the summary of peptide-induced vesicle rupture kinetics based on time-lapse fluorescence microscopy data.

FIG. 8 depicts the electrochemical impedance spectroscopy (EIS) conductance (top) and capacitance (bottom) data for tracking peptide-induced tethered lipid bilayer interactions.

FIG. 9 depicts the CD spectra of peptide 2 with different terminal caps in various solvent environments.

FIG. 10 depicts the QCM-D frequency shift (top) and energy dissipation shift (bottom) data for tracking peptide-induced vesicle rupture of peptides 2 and 4 with different terminal caps.

FIG. 11 depicts the QCM-D frequency shift (top) and energy dissipation shift (bottom) data for tracking peptide-induced vesicle rupture of peptides 6 and 8 with different terminal caps.

FIG. 12 depicts the QCM-D frequency shift (top) and energy dissipation shift (bottom) data for tracking peptide-induced vesicle rupture of peptides 10 and 11 with different terminal caps.

FIG. 13 depicts the summary of peptide-induced vesicle rupture times from QCM-D data for peptides with different terminal caps.

FIG. 14 depicts the time-lapse fluorescence microscopy of peptide-induced vesicle rupture for peptides 2 and 4 with different terminal caps.

FIG. 15 depicts the single-vesicle analysis of peptide-induced vesicle rupture for peptide 2 with different terminal caps based on time-lapse fluorescence microscopy data. A and B correspond to representative kinetic traces for peptide-induced rupture of (A) small and (B) large vesicles.

FIG. 16 depicts the time-lapse fluorescence microscopy of peptide-induced vesicle rupture for peptides 6 and 8 with different terminal caps.

FIG. 17 depicts the time-lapse fluorescence microscopy of peptide-induced vesicle rupture for peptides 10 and 11 with different terminal caps.

### DESCRIPTION

In a first aspect of the invention, there is provided a peptide of 27 amino acid residues in length comprising the following amino acid sequence:

X₁X₂X₃WLX₄X₅X₆WX₇WX₈X₉X₁₀X₁₁X₁₂X₁₃DFX₁₄X₁₅X₁₆LX₁₇X₁₈KX₁₉, wherein:
X₁ is S, G. D, or A;
X₂ is G, D, E, or S;
X₃ is S, D, N, or T;
X₄ is R, H, Y, or W;
X₅ is D, I, T, or E;
X₆ is I, or V;
X₇ is D, E, or N;
X₈ is I, or V;
X₉ is C, or L;
X₁₀ is E, T, S, I, or H;
X₁₁ is I or V;
X₁₂ is L or V;
X₁₃ is S, T, or A;
X₁₄ is K or R;
X₁₅ is T, N, A, V or L;
X₁₆ is W or C;
X₁₇ is K, Q, T, S, or G;
X₁₈ is A or S; and
X₁₉ is L, I, or F (SEQ ID NO: 12)
and wherein:
each amino acid is independently an L- or D-amino acid;
the functional groups of the N- and C-termini of the peptide are:
   (A) unchanged from the standard N- and C-termini functional groups;
   (B) reversed, such that the N-terminus has a carboxylic acid functional group and the C-terminus has an amino functional group;
   (C) the N-terminus and C-terminus both have an amino functional group; or
   (D) the N-terminus and C-terminus both have a carboxylic acid functional group, provided that, when the peptide has the sequence: SGSWLRDVWDWICTVLTDFKTWLQSKL (SEQ ID NO: 2), the functional groups of the N- and C-termini of the peptide are selected from (B) to (D) above only.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

Amino acids can be categorised into different classes depending upon the chemical and physical properties of the amino acid residues. For example, some amino acids are classified based upon their side chains as hydrophilic or polar amino acids, while other amino acids are classified as hydrophobic or nonpolar amino acids. The term “polar amino acid” is a term known to one of skill in the art. Typically a “polar amino acid” refers to a hydrophilic amino acid having a side chain that is charged or uncharged at physiological pH and are generally hydrophilic, meaning that they have an amino acid side chain that is attracted by aqueous solution. Examples of polar amino acids include aspartate, glutamate, glutamine, lysine, arginine, histidine, asparagine, serine, threonine and tyrosine.

Hydrophobic amino acids are known to one of skill in the art. Typically, hydrophobic amino acids can be further classified as having an aliphatic side chain or an aromatic side chain.

Amino acids can be further classified as aliphatic amino acids and aromatic amino acids, which are known to one of skill in the art. Typically, aliphatic amino acids have a side chain containing hydrogen and carbon atoms. Examples of aliphatic amino acids include alanine, isoleucine, proline, and valine. Typically, aromatic amino acids contain a side chain containing an aromatic ring. Examples of aromatic amino acids include phenylalanine, tyrosine, histidine and tryptophan. In some instances, amino acids are classified as small amino acids because they are small in size. Examples of small amino acids include alanine, cysteine, glycine, proline, serine and threonine.

As will be appreciated, an amino acid may be substituted with a different amino acid with similar chemical and physical properties. These substitutions are typically referred to as conservative substitutions. In particular embodiments, a conservative substitution does not alter the structure or function, or both, of a peptide. For example, a hydrophobic amino acid may be substituted for another hydrophobic amino acid, a neutral hydrophilic amino acid may be substituted for another neutral hydrophilic amino acid, an acidic amino acid may be substituted for another acidic amino acid, a basic amino acid may be substituted for another basic amino acid, an aromatic amino acid may be substituted for another aromatic amino acid, etc. Examples of conservative amino acid substitutions are as follows in Table 1.

| Original Residue | Conservative Substitution |
|---|---|
| Ala | Ser, Gly, Thr |
| Arg | Lys, Gln, His, |
| Asn | Asp, Gln, His, Lys, Ser, Thr |
| Asp | Asn, Glu |
| Cys | Ser |
| Gln | Arg, Asn, Glu, His, Lys, Met |
| Gly | Ala, Pro |
| His | Asn, Arg, Gin, Try |
| Ile | Leu, Val, Met |
| Leu | Ile, Met, Phe, Val |
| Lys | Arg, Asn, Gln, Glu |
| Met | Gln, Ile, Leu, Val |
| Phe | Leu, Trp, Tyr, Met |
| Ser | Ala, Asn, Thr |
| Thr | Ser, Ala, Asn |
| Trp | Phe, Tyr |
| Tyr | His, Phe, Trp |
| Val | Ile, Leu, Met |

Table 1

In embodiments of the invention that may be mentioned herein, the peptide may comprise the following amino acid sequence:

X₁X₂X₃WLRX₅X₆WDWX₈CX₁₀X₁₁X₁₂X₁₃DFKX₁₅WLX₁₇X₁₈KX₁₉, wherein:
X₁ is S or G; X₂ is G or D; X₃ is S, D or T; X₅ is D or i; X₆ is I or V; X₈ is I or V; X₁₀ is T or S; X₁₁ is I or V; X₁₂ is L or V; X₁₃ is S, T or A; X₁₅ is T, N or A; X₁₇ is K or S; X₁₈ is A or S; and X₁₉ is L or I (SEQ ID NO: 13) and wherein each amino acid is independently an L- or D-amino acid.

In further embodiments of the invention, the peptide may comprise the following amino acid sequence: SX₂X₃WLRDX₆WDWVCX₁₀VLX₁₃DFKX₁₅WLX₁₇X₁₈KX₁₉, wherein:
X₂ is G or D; X₃ is S, D or T; X₆ is I or V; X₁₀ is T or S; X₁₃ is S or T; X₁₅ is T, N or A; X₁₇ is K or S; X₁₈ is A or S; and X₁₉ is L or I (SEQ ID NO: 14) and wherein each amino acid is independently an L- or D-amino acid.

In yet further embodiments of the invention, the peptide may comprise the following amino acid sequence: SGX₃WLRDIWDWVCX₁₀VLX₁₃DFKTWLSAKX₁₉, where:
X₃ is S, D or T; X₁₀ is T or S; X₁₃ is S or T; and X₁₉ is L or I (SEQ ID NO: 15) and wherein each amino acid is independently an L- or D-amino acid.

In particular embodiments of the invention that may be mentioned herein, the peptide may have a peptide sequence from the following list:

(i)
(SEQ ID NO: 1)
SGSWLRDIWDWICEVLSDFKTWLKAKL;
(ii)
(SEQ ID NO: 2)
SGSWLRDVWDWICTVLTDFKTWLQSKL;
(iii)
(SEQ ID NO: 3)
SGSWLRDVWDWVCTILTDFKNWLTSKL;
(iv)
(SEQ ID NO: 4)
SGSWLRDIWEWVCSILTDFKNWLSAKL;
(v)
(SEQ ID NO: 5)
SDDWLRIIWDWVCSVVSDFKAWLSAKI;
(vi)
(SEQ ID NO: 6)
SGDWLRIIWDWVCSVVSDFKTWLSAKI;
(vii)
(SEQ ID NO: 7)
SDDWLRTIWDWVCSVLADFKAWLSAKI;
(viii)
(SEQ ID NO: 8)
GDDWLHDIWDWVCIVLSDFKTWLSAKI;
(ix)
(SEQ ID NO: 9)
DGNWLYDIWNWVCTVLADFKLWLGAKI;
(x)
(SEQ ID NO: 10)
AESWLWEVWDWVLHVLSDFKTCLKAKF;
and
(xi)
(SEQ ID NO: 11)
GSTWLRDIWDWVCTVLSDFRVWLKSKL,

and wherein each amino acid is independently an L- or D-amino acid.

The amino acid sequences disclosed herein may be one of the following:
each amino acid in each sequence is an L-amino acid;
each amino acid in each sequence is a D-amino acid; or
the amino acids in each sequence are a mixture of L- and D-amino acids.

In particular embodiments of the invention that may be mentioned herein, each amino acid in each sequence is a D-amino acid or, more particularly, each amino acid in each sequence is an L-amino acid.

It has been surprisingly found that modifying the N- and C-termini of the peptides (so that they become a carboxylic acid or amino group, respectively) may result in a compound (peptide) that has better properties (e.g. in terms of potency and/or selectivity) than the original peptide has. Without wishing to be bound by theory, it is believed that simply reversing the N- and C-termini's positions does not significantly affect the properties of the resulting compound. As such, in particular embodiments of the invention that may be disclosed herein, the functional groups of the N- and C-termini of the peptides disclosed herein may be ones in which:
(AI) the N-terminus and C-terminus both have an amino functional group; or
(AII) the N-terminus and C-terminus both have a carboxylic acid functional group.

In certain embodiments, the amino acid sequences described herein may be linked (directly or indirectly) to a peptide tag (e.g., a HIS-tag or FLAG-tag). In some embodiments, a linker sequence (e.g., a 1-5, 1-10 or 5-10 amino acid linker, such as a glycine linker) may be used to link a peptide tag to the amino acid sequences described herein. In other embodiments, a peptide tag is linked directly to an amino acid sequence described herein. The linkage between the peptide tag and the amino acid sequence may be covalent or non-covalent

In a specific embodiment, the amino acid sequence described herein may comprise one, two, or more amino acid derivatives. For example, in a specific embodiment, an amino acid sequence described herein may comprise one, two, or more non-naturally occurring or non-genetically encoded amino acids. Non-limiting examples of non-genetically encoded amino acids include α-amino hexanoic acid, α-amino valeric acid, 1,2,3,4-tetrahydro-isoquinoline-3-carboxylic acid, 2,3-diaminobutyric acid, 2,3-diaminopropionic acid, 2,4-diamino butyric acid, 2-fluorophenylalanine, 3-bezothienyl alanine, 3-fluorophenylalanine, 4-chlorophenylalanine, 4-fluorophenylalanine, α-2-thienylalanine, α-alanine, α-aminoisobutyric acid, citrulline, cyclohexylalanine, phenylglycine, homoarginine, homocysteine, homoserine, methionine sulfoxide, N-acetyl lysine, N-aminophenylalanine, N-methylglycine, N-methylisoleucine, N-methylvaline, naphthylalanine, norleucine, omithine, penicillamine, pyridylalanine, t-butylalanine, and t-butylglycine.

In another aspect of the invention, the peptides disclosed hereinbefore may be pegylated. That is, there is provided a pegylated peptide comprising a peptide of 27 amino acid residues in length as described hereinbefore linked to one, two or more polyethylene glycol (PEG) polymers, optionally wherein:
(aa) the one, two or more PEG polymers may be linked to the N-terminus and/or C-terminus of the peptide; and/or
(ab) each of the one, two or more PEG polymers may have a molecular weight range of from 500 to 5,000 Daltons;
(ac) each of the one, two or more PEG polymers may be branched or unbranched.

For the avoidance of doubt, reference to the “peptides disclosed herein” includes reference to the pegylated form of the peptides unless specifically stated otherwise. In a specific embodiment, the PEG polymer(s) may have a molecular weight of from 500 to 1,000 Daltons, from 500 to 2,000 Daltons, from 500 to 3,000 Daltons, from 500 to 4,000 Daltons, or from 500 to 5,000 Daltons. In a specific embodiment, the PEG polymer(s) may have the molecular weight range of from 1,000 to 5,000 Daltons, from 2,000 to 5,000 Daltons, from 3,000 to 5,000 Daltons, or from 4,000 to 5,000 Daltons. In another embodiment, the PEG polymer(s) may have the molecular weight range of from 500 to 5,000 Daltons. The PEG polymer(s) may be branched, non-branched or forked. In certain embodiments, one, two or more of the PEG polymers may be branched. In some embodiments, one, two or more of the PEG polymers may be non-branched. In certain embodiments, one, two or more of the PEG polymers may be forked. As will be appreciated, when there are two PEG polymers, one may be unbranched and the other may be branched.

The peptides described herein may be linked to one, two, or more PEG polymers at, for example, the N-terminus of the peptide, the C-terminus of the peptide, a lysine residue in the peptide, or an arginine residue in the peptide. In a specific embodiment, the one, two, or more PEG polymers may be linked to the N-terminus of the peptide. In another specific embodiment, the one, two, or more PEG polymers may be linked to the C-terminus of the peptide. In another specific embodiment, the one, two, or more PEG polymers may be linked to a lysine residue in the peptide. In another specific embodiment, the one, two, or more PEG polymer may be linked to an arginine residue in the peptide. In certain embodiments, the peptide described herein may be linked to two or more PEG polymers at two or more of the following: the N-terminus of the peptide, the C-terminus of the peptide, a lysine residue in the peptide, and/or an arginine residue in the peptide.

The pegylation of a peptide described herein may increase the half-life of the peptide in vivo as assessed by techniques known to one of skill in the art. See, e.g., Bird, Gregory H., et al. “Hydrocarbon double-stapling remedies the proteolytic instability of a lengthy peptide therapeutic.” Proceedings of the National Academy of Sciences 107.32 (2010): 14093-14098, which is incorporated herein by reference, for a description of techniques for assessing the half-life of a peptide. For example, one non-human animal (e.g., mice) may be administered a peptide of a certain concentration and another non-human animal of the same species (e.g., mice) may be administered the same peptide that is pegylated or includes a hydrophilic polymer. At various time points after administration of the peptides, blood may be drawn from each animal and the concentration of peptide in the blood from each animal at each time point may be assessed by, e.g., liquid chromatography/mass spectrometry (LC/MS). For example, the pegylation of a peptide described herein may increase the half-life of the peptide in vivo by 2 to 5 times, 2 to 10 times, 2 to 20 times, 2 to 25 times, 2 to 50 times, 2 to 75 times, or 2 to 100 times as assessed by techniques known to one of skill in the art.

In embodiments disclosed herein, the peptide described herein may be in the form of an amphipathic helix. In examples disclosed herein, the peptide may be weakly helical. That is, the peptides described herein may have a fractional helicity of from 0.1 to 0.5 in distilled water, and from 0.1 to 0.6 in phosphate-buffered saline. Fractional helicity may be determined using any technique known to one of skill in the art or described herein. For example, circular dichroism may be used as follows: CD experiments are conducted on a AVIV Model 420 spectrometer (AVIV Biomedical, Lakewood, NJ, USA) using quartz curvette with a 1 mm path length (Hellma). Spectral data are collected with a step size of 0.5 nm and averaging time of 4 seconds. All spectra are recorded at 25° C. from 190 to 260 nm using a bandwidth of 1-nm and averaged over three scans. The CD spectra are recorded before and after the addition of 2.5 mM POPC lipid vesicles to 50 μM peptide. Baseline scans in buffer only or liposomes only are also performed using the same instrument settings, and this contribution was subtracted from respective data scans with peptides. The corrected spectra are expressed in mean residue molar ellipticity (O), and the fractional helicity of peptides is calculated as follows:

fH=([Θ]₂₂₂−3,000)/(−36,000−3000), where [Θ]₂₂₂ is the molar ellipticity at 222 nm.

See, e.g., J. D. Morrisett, J. S. David, H. J. Pownall, A. M. Gotto Jr, Interaction of an apolipoprotein (apoLP-alanine) with phosphatidylcholine. Biochemistry 12, 1290-1299 (1973), which is incorporated herein for a description of techniques for determining fractional helicity.

In embodiments that may be mentioned herein, the peptides may undergo significant helical induction (e.g., a greater than 20% increase) in zwitterionic lipid vesicles. In other words, there is a helicity increase from its secondary structure state in solution (in PBS) to its secondary structure state when inserted into lipid membranes (in PBS). If the helicity of a peptide becomes greater in the lipid membrane, it prefers to associate into the lipid membrane because it is more stable. In embodiments disclosed herein, the peptide may undergoes a 21% to 50% or 21% to 30% increase helical induction in zwitterionic lipid vesicles. In a specific embodiment, the peptides described herein may be folded into an alpha-helical state in zwitterionic lipid vesicles. In embodiments, the helical induction may be determined according to circular dichroism. In other embodiments, the helical induction may be determined using a protein algorithm or FTIR spectroscopy. See, e.g., Haris, Parvez I., and Dennis Chapman. “The conformational analysis of peptides using Fourier transform IR spectroscopy.” Biopolymers 37.4 (1995): 251-263 for a description of the FTIR spectroscopy method.

Without being bound by any particular theory, the peptides described herein may act as a membrane curvature sensor, which is preferentially active against highly curved membranes, such as, for example, small, enveloped viruses. For example, the peptides disclosed herein may exhibit strong partitioning into zwitterionic lipid bilayers. In an embodiment of the invention, the peptides described herein may have a lipid-water partition coefficient (also known as partition constant) of approximately 10⁵ for high-curvature, zwitterionic lipid vesicles of less than 200 nm in diameter. In embodiments of the invention that may be mentioned herein, the peptide described herein may have negligible partitioning into larger vesicles. Negligible partitioning means a lipid-water partition coefficient of less than 1×10⁴. The partitioning of the peptide into zwitterionic 1-palmitoyl-2-oleoyl-sn-glycero-3-phosphocholine lipid bilayers may be determined according to fluorescence spectroscopy.

In embodiments of the invention that may be mentioned herein, the peptides described herein may have a low tendency to aggregate. The tendency to aggregate can be determined by absorbance measurements at 280 nm wavelength, which can determine the molar concentration of peptide based on the molar extinction coefficient that is defined by the number of tryptophans, tyrosines, and phenylalanines. A low tendency to aggregate (in other words, good dispersion) means that the peptide reconstitutes well in an aqueous solution (e.g., distilled water) and is defined as >60%, where % is defined as experimental molar mass measured by absorbance measurements/theoretical molar mass based on dry mass and molecular weight×100%. In contrast, a high tendency to aggregate (in other words, bad dispersion) is defined as <60%, where % is defined as experimental molar mass measured by absorbance measurements divided by the theoretical molar mass, calculated based on the dry mass and molecular weight of the peptide, ×100%.

References herein (in any aspect or embodiment of the invention) to the peptide sequences or simply peptides, include references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a peptide disclosed herein with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a peptide disclosed herein in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by a peptide disclosed herein are any solvates of the peptides and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of the peptides disclosed herein as defined herein include ester derivatives and/or derivatives that have, or provide for, the same biological function and/or activity as any relevant compound of the invention. Thus, for the purposes of this invention, the term also includes prodrugs of the peptides disclosed herein.

The term “prodrug” of a relevant peptide disclosed herein includes any peptide that, following oral or parenteral administration, is metabolised in vivo to form the parent compound (peptide) in an experimentally-detectable amount, and within a predetermined time (e.g. within a dosing interval of between 6 and 24 hours (i.e. once to four times daily)).

Prodrugs of the peptides disclosed herein may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include the peptides disclosed herein wherein a hydroxyl, amino, sulfhydryl, carboxyl or carbonyl group in a peptide disclosed herein is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

The peptides disclosed here, as well as pharmaceutically acceptable salts, solvates and pharmaceutically functional derivatives of such compounds are, for the sake of brevity, hereinafter referred to together as the “peptides disclosed herein”.

The peptides disclosed herein may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Further embodiments of the invention that may be mentioned include those in which the peptides disclosed herein are isotopically labelled. However, other, particular embodiments of the invention that may be mentioned include those in which the peptides disclosed herein are not isotopically labelled.

The term “isotopically labelled”, when used herein includes references to the peptides disclosed herein in which there is a non-natural isotope (or a non-natural distribution of isotopes) at one or more positions in the compound. References herein to “one or more positions in the compound” will be understood by those skilled in the art to refer to one or more of the atoms of the peptides disclosed herein. Thus, the term “isotopically labelled” includes references to the peptides disclosed herein that are isotopically enriched at one or more positions in the compound.

The isotopic labelling or enrichment of the peptides disclosed herein may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the peptides disclosed herein is labelled or enriched with a radioactive or nonradioactive isotope, peptides disclosed herein that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

The peptides described herein can be produced by any method known in the art for the synthesis of peptides, in particular, by chemical synthesis or recombinant expression techniques, or by any method described herein. The methods provided herein encompass, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

The peptides described herein may be produced synthetically. The peptides described herein may be prepared using conventional step-wise solution or solid phase synthesis (see, e.g., Chemical Approaches to the Synthesis of Peptides and Proteins, Williams et al., Eds., 1997, CRC Press, Boca Raton Fla., and references cited therein; Solid Phase Peptide Synthesis: A Practical Approach, Atherton & Sheppard, Eds., 1989, IRL Press, Oxford, England, and references cited therein). Alternatively, the anti-infective peptides described herein may be prepared by way of segment condensation, as described, for example, in Liu et al., 1996, Tetrahedron Lett. 37(7):933-936; Baca, et al, 1995, J. Am. Chem. Soc. 117: 1881-1887; Tam et al, 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, Proc. Natl. Acad. Sci. USA 91:6584-6588; Yamashiro and Li, 1988, Int. J. Peptide Protein Res. 31:322-334. Other methods useful for synthesizing the anti-infective peptides described herein are described in Nakagawa et al, 1985, J. Am. Chem. Soc. 107:7087-7092.

The peptides described herein may be generated by standard F-moc solid phase synthesis. For example, the (anti-infective) peptide is generated by standard F-moc solid phase synthesis and purified by reverse-phase, high-performance liquid chromatography. The molecular weight of the purified peptide is determined by MALDI mass spectrometry. Lyophilized peptide samples are kept at −20° C. for long-term storage. Aliquots are prepared by solubilizing the peptide in deionized water at room temperature to a stock concentration of 2 mg/ml, and then stored at −20° C. until use. The molar concentration of the peptide in solution is estimated by taking into account the molar extinction coefficient of each aromatic tryptophan residue present in the amino acid sequence, as determined by absorbance measurements at 280 nm.

In certain embodiments, the peptides disclosed herein may be recombinantly produced. In a specific embodiment, provided herein is a nucleic acid sequence encoding a peptide described herein. Due to the degeneracy of the genetic code, any nucleic acid that encodes a peptide described herein is encompassed herein.

As used herein, the term “nucleic acid” is intended to include DNA molecules (e.g., cDNA or genomic DNA) and RNA molecules (e.g., mRNA) and analogs of the DNA or RNA generated using nucleotide analogs. The nucleic acid can be single-stranded or double-stranded.

Also provided herein are nucleic acid sequences capable of hybridizing to a nucleic acid sequence encoding a peptide disclosed herein. In certain embodiments, provided herein are nucleic acids capable of hybridizing to a fragment of a nucleic acid encoding a peptide disclosed herein. In other embodiments, provided herein are nucleic acids capable of hybridizing to the full length of a nucleic acid encoding a peptide disclosed herein. General parameters for hybridization conditions for nucleic acids are described in Sambrook et al., Molecular Cloning—A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

In some embodiments, a nucleic acid sequence encoding a peptide disclosed herein may be isolated. In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA molecule, can be substantially free of other cellular material, or culture medium when produced by recombinant techniques, or substantially free of chemical precursors or other chemicals when chemically synthesized. The term “substantially free of cellular material” includes preparations of nucleic acid sequence in which the nucleic acid sequence is separated from cellular components of the cells from which it is isolated or recombinantly produced. Thus, the nucleic acid sequence that is substantially free of cellular material includes preparations of the nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

Provided herein are vectors, including expression vectors, containing a nucleic acid sequence encoding a peptide described herein. In a specific embodiment, the vector is an expression vector that is capable of directing the expression of a nucleic acid sequence encoding a peptide described herein. Non-limiting examples of expression vectors include, but are not limited to, plasmids and viral vectors, such as replication defective retroviruses, adenoviruses, adeno-associated viruses and baculoviruses. Expression vectors also may include, without limitation, transgenic animals and non-mammalian cells/organisms, e.g., mammalian cells/organisms that have been engineered to perform mammalian N-linked glycosylation.

An expression vector comprises a nucleic acid sequence encoding a peptide described herein and in a form suitable for expression of the nucleic acid in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid sequence of interest is linked to the regulatory sequence(s) in a manner which allows for expression of the nucleic acid sequence (e.g., in an in vitro transcription/translation system or in a host cell when the vector is introduced into the host cell). Regulatory sequences include promoters, enhancers and other expression control elements {e.g., polyadenylation signals). Regulatory sequences include those which direct constitutive expression of a nucleic acid sequence in many types of host cells, those which direct expression of the nucleic acid sequence only in certain host cells (e.g., tissue-specific regulatory sequences), and those which direct the expression of the nucleic acid sequence upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid sequence and the progeny or potential progeny of such a cell. Progeny of such a cell may not be identical to the parent cell transformed or transfected with the nucleic acid sequence due to mutations or environmental influences that may occur in succeeding generations or integration of the nucleic acid sequence into the host cell genome. In specific embodiments, the host cell is a cell line.

Expression vectors can be designed for expression of an anti-infective peptide described herein using prokaryotic (e g., E. coli) or eukaryotic cells (e.g., insect cells (using baculovirus expression vectors, see, e.g., Treanor et al., 2007, JAMA, 297(14): 1577-1582 incorporated by reference herein in its entirety), yeast cells, plant cells, algae, avian, or mammalian cells). Examples of yeast host cells include, but are not limited to 5. pombe and 5. cerevisiae. An example of avian cells includes, but is not limited to EB66 cells. Examples of mammalian host cells include, but are not limited to, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TGI.7 cells, AF-2 clone 9B5 cells, AF-2 clone 9B5 cells, J558L cells, MOPC 315 cells, MPC-11 cells, NCI-H929 cells, NP cells, NSO/1 cells, P3 NS1 Ag4 cells, P3/NSI/I-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include 5/9, 5/21, Trichoplusia ni, Spodoptera frugiperda and Bombyx mori. In a particular embodiment, a mammalian cell culture system (e.g. Chinese hamster ovary or baby hamster kidney cells) is used for expression of an anti-infective peptide. In another embodiment, a plant cell culture system is used for expression of an anti-infective peptide. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. The host cells comprising a nucleic acid sequence that encodes an anti-infective peptides described herein can be isolated, i.e., the cells are outside of the body of a subject. In certain embodiments, the host cells are engineered to express a nucleic acid sequence that encodes an anti-infective peptide described herein. In specific embodiments, the host cells are cells from a cell line.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning—A Laboratory Manual, 2nd Edition, Cold Spring Harbor Press, New York, and other laboratory manuals. In certain embodiments, a host cell is transiently transfected with an expression vector containing a nucleic acid sequence encoding an anti-infective peptide. In other embodiments, a host cell is stably transfected with an expression vector containing a nucleic acid sequence encoding a peptide as described herein.

For stable transfection of mammalian cells, it is known that, depending upon the expression vector and transfection technique used, only a small fraction of cells may integrate the foreign DNA into their genome. In order to identify and select these integrants, a nucleic acid sequence that encodes a selectable marker (e.g., for resistance to antibiotics) is generally introduced into the host cells along with the nucleic acid sequence of interest. Examples of selectable markers include those which confer resistance to drugs, such as G418, hygromycin and methotrexate. Cells stably transfected with the introduced nucleic acid sequence can be identified by drug selection (e.g., cells that have incorporated the selectable marker gene will survive, while the other cells die).

As an alternative to recombinant expression of a peptide described herein using a host cell, an expression vector containing a nucleic acid sequence encoding an anti-infective peptide can be transcribed and translated in vitro using, e.g., T7 promoter regulatory sequences and T7 polymerase. In a specific embodiment, a coupled transcription/translation system, such as Promega TNT™, or a cell lysate or cell extract comprising the components necessary for transcription and translation may be used to produce an anti-infective peptide.

Once a peptide according to the invention has been produced, it may be isolated or purified by any method known in the art for isolation or purification of a protein, for example, by chromatography {e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a peptide as described herein. In one embodiment, the method comprises culturing a host cell containing a nucleic acid sequence encoding a peptide as described herein in a suitable medium such that the peptide is produced. In some embodiments, the method further comprises isolating the peptide from the medium or the host cell.

As will be appreciated, the peptides disclosed herein are particularly useful in the treatment of a range of infectious diseases. Thus, in a further aspect of the invention, there is disclosed a pharmaceutical composition comprising a peptide and/or a pegylated peptide as disclosed herein and a pharmaceutically acceptable carrier. In embodiments of the invention, the carrier may be an aqueous solution (e.g. sterile and/or distilled water).

In embodiments of the invention, the composition may further comprise one or more pharmaceutically acceptable excipients and adjuvants.

The peptides and/or pharmaceutical composition disclosed herein may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound (peptide) in a pharmaceutically acceptable dosage form. Particular modes of administration that may be mentioned include oral, intravenous, cutaneous, subcutaneous, nasal, intramuscular or intraperitoneal administration. In particular embodiments of the invention, the pharmaceutical composition disclosed herein may be formulated for subcutaneous, intravenous or intraperitoneal administration.

The peptides disclosed herein will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of a peptide disclosed herein in any pharmaceutical formulation used in accordance with the present invention will depend on various factors, such as the severity of the condition to be treated, the particular patient to be treated, as well as the compound(s) which is/are employed. In any event, the amount of a peptide disclosed herein in the formulation may be determined routinely by the skilled person.

For example, a solid oral composition such as a tablet or capsule may contain from 1 to 99% (w/w) active ingredient (e.g., peptide disclosed herein); from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant- and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, the peptides disclosed herein may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a peptide disclosed herein.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

In a further aspect of the invention, there is disclosed a vial containing a pharmaceutical composition as described hereinbefore. As will be appreciated, the composition may be initially prepared at the site of manufacture and then subjected to lyophilization for transport and storage. Thus, the vial comprising the pharmaceutical composition may be one in which the pharmaceutical composition is lyophilized.

As noted above, the peptides disclosed herein may be suitable for use in the treatment of various disorders. Thus, in a further aspect of the invention, there is disclosed a peptide and pegylated peptide as described hereinbefore for use in medicine. Further aspects that are disclosed include:
(AA) a method of treating or preventing a viral infection in a subject, the method comprising administering a pharmaceutically effective amount of a peptide or a pegylated peptide as described hereinbefore;
(AB) a peptide or a pegylated peptide as described herein for use in treating or preventing a viral infection; and
(AC) use of a peptide or a pegylated peptide as described herein in the preparation of a medicament for treating or preventing a viral infection.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The peptides and pegylated peptides disclosed herein may be used to treat any suitable viral infection. Examples of suitable viral infections include, but are not limited to, viruses of the coronaviridae or, more particularly, flaviviridae, togaviridae, filoviridae, arenaviridae, poxviridae, bunyaviridae, and retroviridae families.

In particular embodiments of the invention, the viral infection may be a mosquito-borne virus. For example, the viral infection may be selected from one or more of the group consisting of dengue, Zika, yellow fever, West Nile, and Chikungunya.

In further embodiments of the invention the viral infection may be selected from one or more of the group consisting of HCV, HDV, JEV, COVID19, SARS, MERS-CoV, MERS, SARS-CoV-2, alphacoronavirus, betacoronavirus, gammacoronavirus or, more particularly, dengue, Chikungunya virus, Ebola, HIV, West Nile, Zika, yellow fever, and influenza.

Further aspects and embodiments of the invention may be found in the following non-limiting examples.

### EXAMPLES

### Materials

The peptides were commercially synthesized and all reagents were purchased from Sigma-Aldrich unless otherwise noted. Titanium oxide-coated sensor chips were purchased from Biolin Scientific. Glass coverslips and microfluidic flow cell were purchased from ibidi GmbH. Poly(L-lysine)-grafted poly(ethylene glycol)-biotin (PLL-g-PEG(-biotin)) was purchased from SuSoS AG. Vero cells (catalogue no. CCL-81) was purchased from ATCC. 4G2, a mouse anti-DENV E protein monoclonal antibody (1:2,000 dilution) was purchased from Integrated BioTherapeutics. Goat anti-mouse IgG (H+L)-HRP conjugate (1:2,000 dilution, catalogue no. 170-6516) was purchased from Bio-Rad.

### Example 1. Preparation of Peptides

A new therapeutic strategy called Lipid Envelope Antiviral Disruption (LEAD) was developed to thwart mosquito-borne viral infections. The rationale of the LEAD approach is that therapeutic reduction of the concentration of infectious virions can mitigate viral spread and ameliorate disease symptoms, and the challenge lies in identifying drug candidates that can accomplish this task in vivo. As Zika virus infection is associated with viral replication in the brain, we focused on engineering a series of 27-mer, amphipathic, α-helical (AH) peptides. The AH peptides were derived from the first 27 amino acids of hepatitis C virus NS5A protein and share a common region of amino acid sequence with the C5A peptide described above. The series of 27-mer peptides and peptide name designations and amino acid sequences are listed in Table 1.

TABLE 1
Peptide name designations and amino acid
sequences.

| # | Amino Acid Sequence |
|---|---|
| 1 | SGSWLRDIWDWICEVLSDFKTWLKAKL |
| 2 | SGSWLRDVWDWICTVLTDFKTWLQSKL |
| 3 | SGSWLRDVWDWVCTILTDFKNWLTSKL |
| 4 | SGSWLRDIWEWVCSILTDFKNWLSAKL |
| 5 | SDDWLRIIWDWVCSVVSDFKAWLSAKI |
| 6 | SGDWLRIIWDWVCSVVSDFKTWLSAKI |
| 7 | SDDWLRTIWDWVCSVLADFKAWLSAKI |
| 8 | GDDWLHDIWDWVCIVLSDFKTWLSAKI |
| 9 | DGNWLYDIWNWVCTVLADFKLWLGAKI |
| 10 | AESWLWEVWDWVLHVLSDFKTCLKAKF |
| 11 | GSTWLRDIWDWVCTVLSDFRVWLKSKL |

Peptides were synthesized by standard F-moc solid-phase synthesis and purified to >90% by reverse-phase, high-performance liquid chromatography (Anaspec Corporation). The molecular weight of the purified peptides was verified by matrix-assisted laser desorption/ionization mass spectrometry. Lyophilized peptide samples were kept at −20° C. for long-term storage. For in vitro experiments, the aliquots were prepared by solubilizing the peptides in deionized water (8% dimethyl sulfoxide, DMSO) at room temperature to a stock concentration of 2 mg ml⁻¹ (˜608 μM), and then stored at −20° C. until the experiment. Absorbance measurements at a wavelength of 280 nm were conducted to determine the molar concentration of peptides in stock solutions, and the peptides were subsequently diluted with appropriate media (aqueous buffer or cell culture medium) depending on the experiment.

### Example 2. Characterization of Peptides

### CD Spectroscopy

CD spectroscopy measurements were performed on an AVIV Model 420 spectrometer (AVIV Biomedical) using a quartz cuvette with a path length of 1 mm (Hellma). The experiments were conducted at 25° C. and data were collected in the spectral range from 190 to 260 nm. The bandwidth was 1 nm and three scans were conducted per run (4 s averaging). The CD spectra were recorded before and after adding 2.5 mM POPC liposomes to 50 μM peptide. Baseline scans in buffer only or liposomes only were performed using the same instrumental settings, and the baseline-adjusted spectra are reported in mean residue molar ellipticity (O) units.

### Results and Discussion

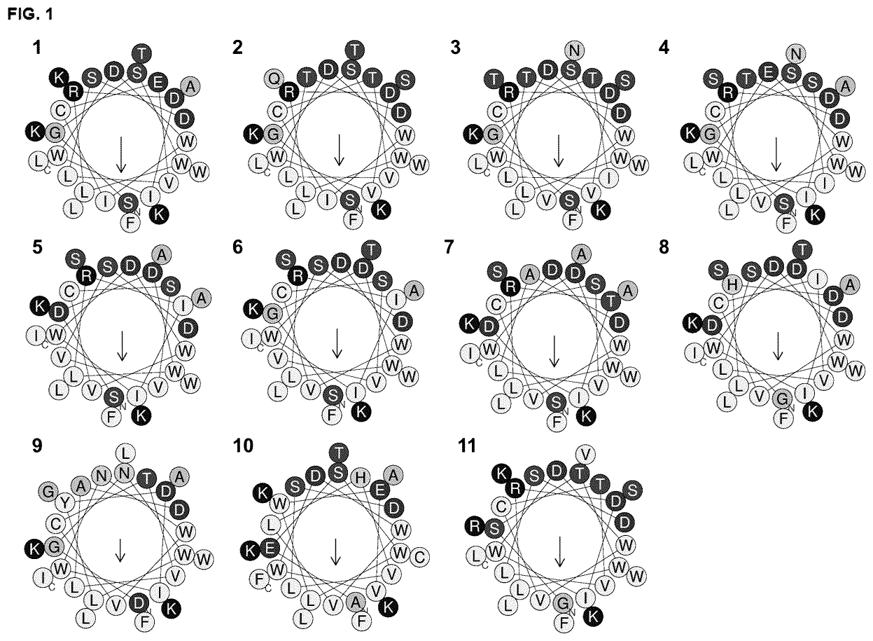

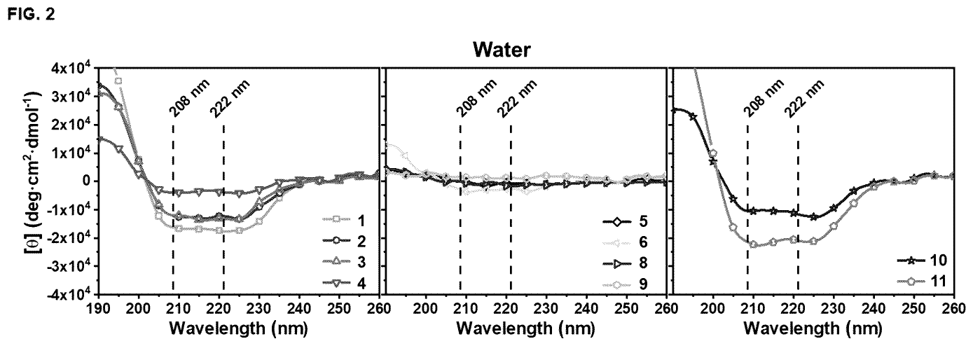

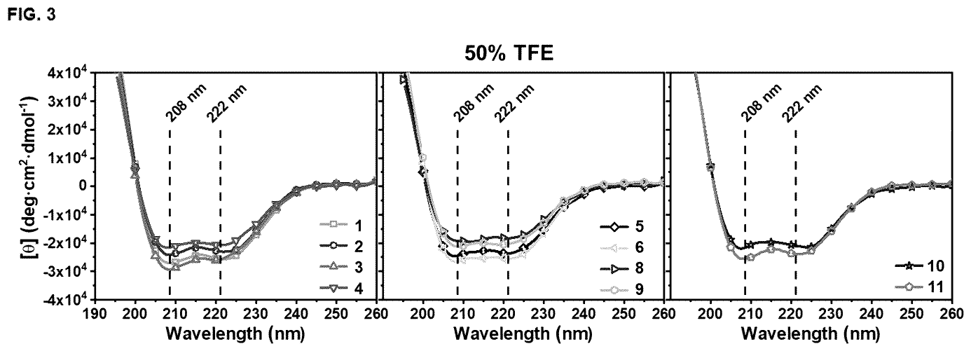

The peptides had varying degrees of amphipathic disposition based on amino acid sequence (FIG. 1) and different degrees of α-helicity in water (FIG. 2). Notably, all peptides had extensive α-helical character in 50% TFE, which indicates high helix stability or a high propensity to undergo a coil-to-helix transition in hydrophobic environments, depending on the case (FIG. 3). The latter feature is advantageous for membrane-disruptive peptides.

### Example 3. In Vitro Antiviral Activity of Peptides

### QCM-D Monitoring

QCM-D measurements were performed using a Q-Sense E4 instrument (Biolin Scientific). All measurements were performed on titanium oxide-coated sensor chips. Time-dependent shifts in the resonance frequency (Δf) and energy dissipation (ΔD) signals were monitored to track adsorption processes, including AH peptide-mediated liposome rupture. After stabilizing the measurement signals, 0.125 mg ml⁻¹ POPC liposomes were added, followed by a washing step. Then, 13 μM AH peptide solution was added at a volumetric rate of 50 μl min⁻¹, and the measurement data were collected using the QSoft (Biolin Scientific) software program. QTools (Biolin Scientific) and OriginPro 8.5 (OriginLab) software programs were used for data processing. The presented data are reported from the fifth overtone.

### Time-Lapse Fluorescence Microscopy Imaging

Highly parallel measurements of peptide-induced liposome rupture were conducted by time-lapsed epifluorescence microscopy imaging. An Eclipse Ti-E inverted microscope (Nikon) with a high-pressure mercury lamp, a CFI Plan Apochromat TIRF×60 oil-immersion objective (NA 1.49; Nikon) and an Andor iXon3 897 EMCCD camera (Andor Technology), was utilized for measurements. The samples were illuminated with a mercury lamp (Intenslight C-HGFIE) using a TRITC filter set. Briefly, glass coverslips were assembled into a microfluidic flow cell, and coated with a thin layer of PLL-g-PEG(-biotin), followed by tethering of 0.025 μg ml⁻¹ liposomes via biotin-neutravidin coupling. Peptide was then added at a volumetric rate of 100 μl min⁻¹, and micrographs were recorded every 5-30 s depending on the particular experiment. The initial time was defined by when the peptide solution entered the channel inlet. Data processing and analysis were conducted using ImageJ (National Institutes of Health) and Python(x,y) software packages. The diameter of individual liposomes was estimated based on fluorescence intensity values.

### EIS

The EIS measurement platform consisted of gold electrodes that were supplied by SDx Tethered Membranes (Roseville, New South Wales, Australia) and coated with 10 mol % tether (benzyl disulfide octo-ethylene glycol phytanyl) and 90 mol % spacer (hydroxyl terminated benzyl disulfide tetra-ethylene glycol) molecules dispersed in ethanol. The assembled tethaPlate (SDx Tethered Membranes) contained six flow cells with a 2.1 mm² electrode surface area per cartridge, and the formation of tethered bilayer lipid membranes (tBLMs) on the sensor surfaces was initiated by the addition of 8 μl of 3 mM DOPC lipid in ethanol. After 2 min of incubation, the flow cell was rapidly rinsed with 10 mM Tris [pH 7.5, 150 mM NaCl] buffer (3 cycles×100 μl per cycle) in order to promote tBLM formation. Afterward, 100 μl of the appropriate test sample was injected into each well. TethaPod and tethaPatch units from SDx Tethered Membranes were used as the membrane conductance and capacitance reader and as the potentiostat connectivity unit, respectively. All measurements were collected under ambient room-temperature conditions (−25° C.) and analyzed using the tethaQuick software program (SDx Tethered Membranes).

### Plaque Reduction Neutralization Assay

Vero cells were seeded at 1×10⁵ cells per well and incubated overnight. Twofold serial dilutions of peptide were incubated with 200-300 p.f.u. of virus for 1 h at 37° C. Cells were infected with the virus-peptide mixture for 1 h at 37° C. The inoculum was removed and 0.8% (wt/vol) methylcellulose in medium supplemented with 1% FBS was added. After 3-5 days incubation, plates were fixed and analysed for plaques by immunostaining (DENV). For immunostaining, the cells were fixed, followed by incubation with 4G2, a mouse anti-DENV E protein monoclonal antibody (1:2,000 dilution), for 16-20 h. After addition of a secondary antibody, goat anti-mouse IgG (H+L)-HRP conjugate (1:2,000 dilution), the TrueBlue peroxidase substrate (KPL) was added and the optical density was measured at a wavelength of 450 nm. For crystal violet staining, cells were fixed with fixing solution (5% glutaric dialdehyde in Dulbecco's PBS) followed by staining with 0.1% crystal violet in fixing solution. The IC₅₀ value was calculated by comparing the relative number of plaques in peptide-treated samples to virus-only samples using a four-parameter logistic curve fit.

### Results and Discussion

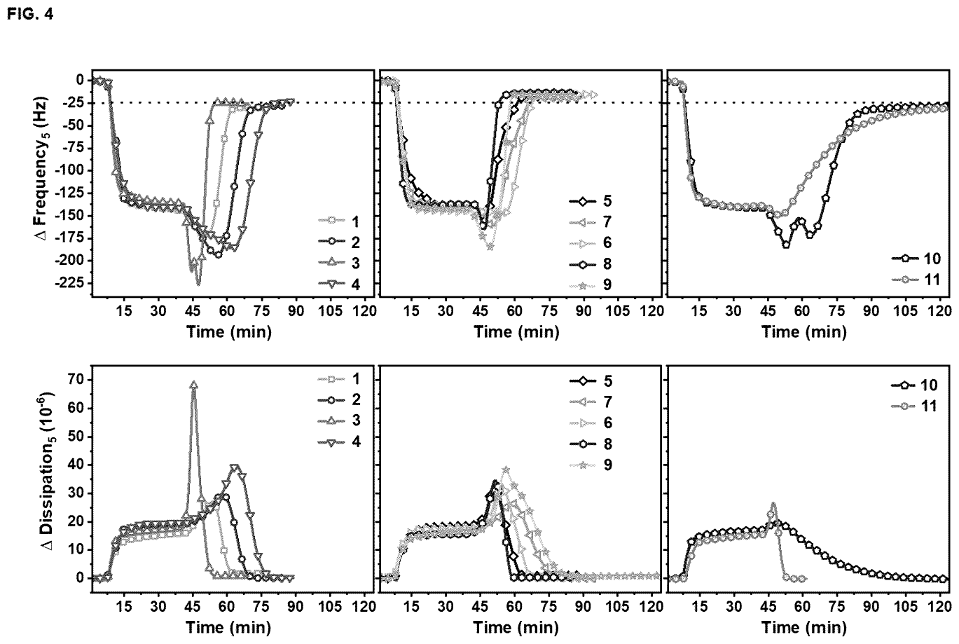

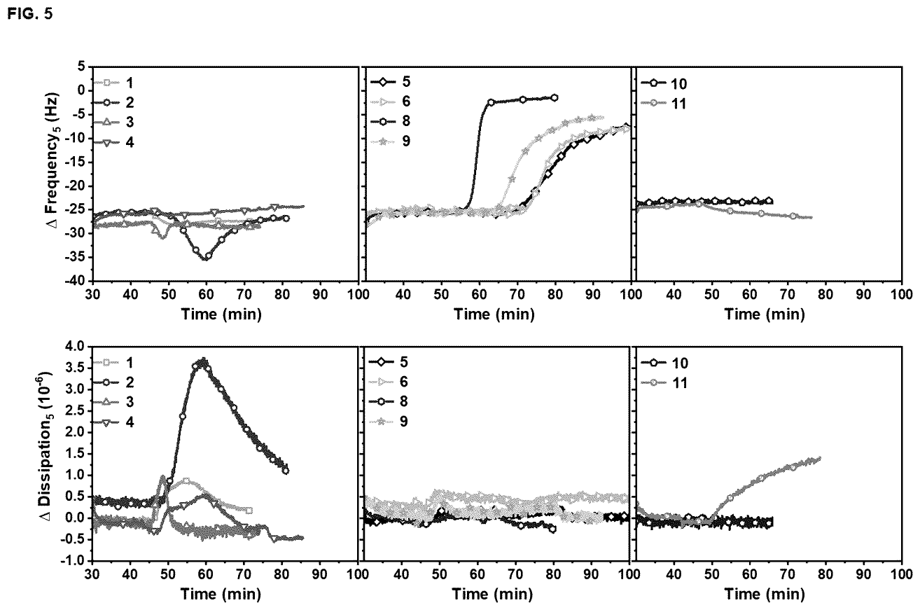

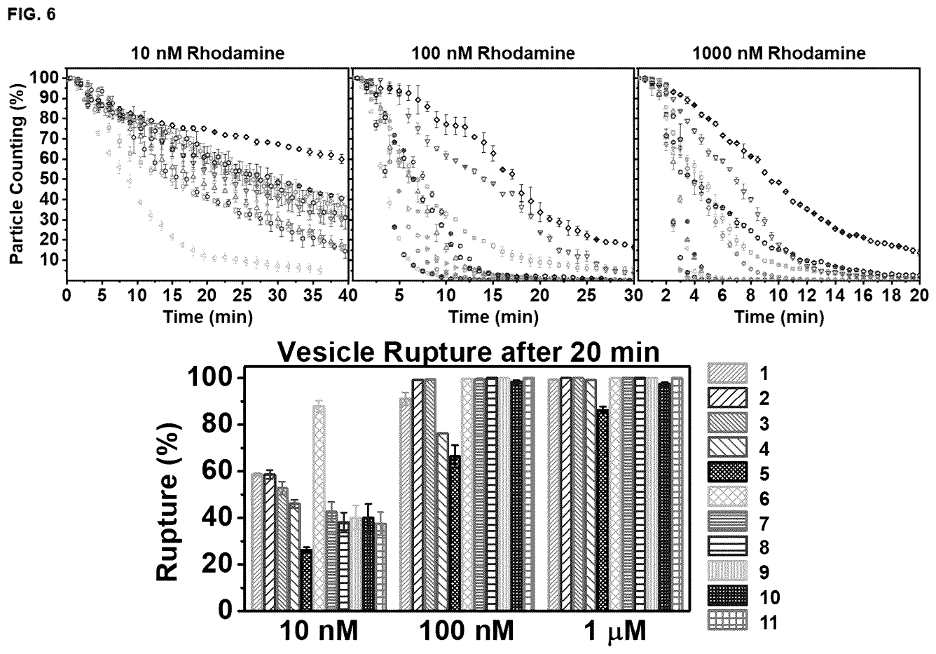

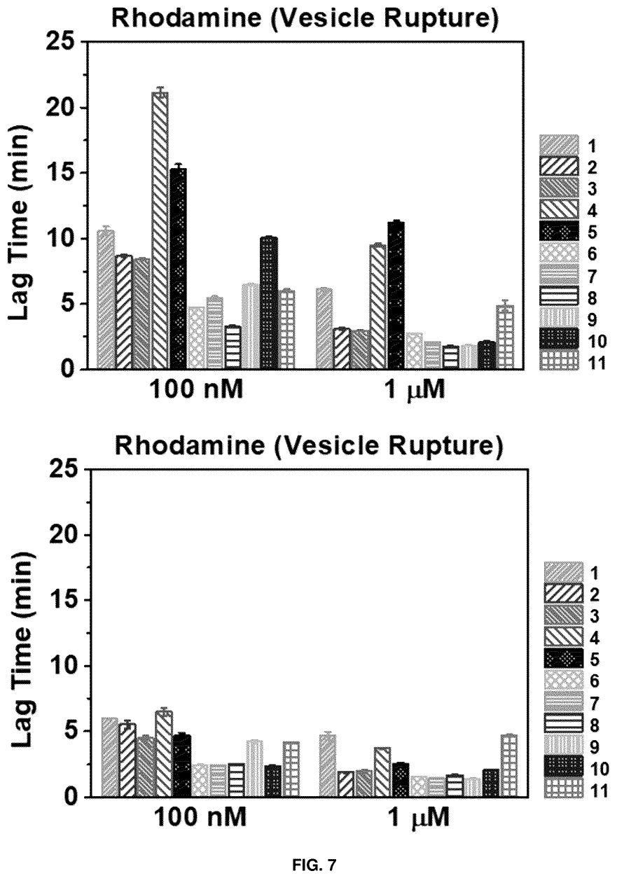

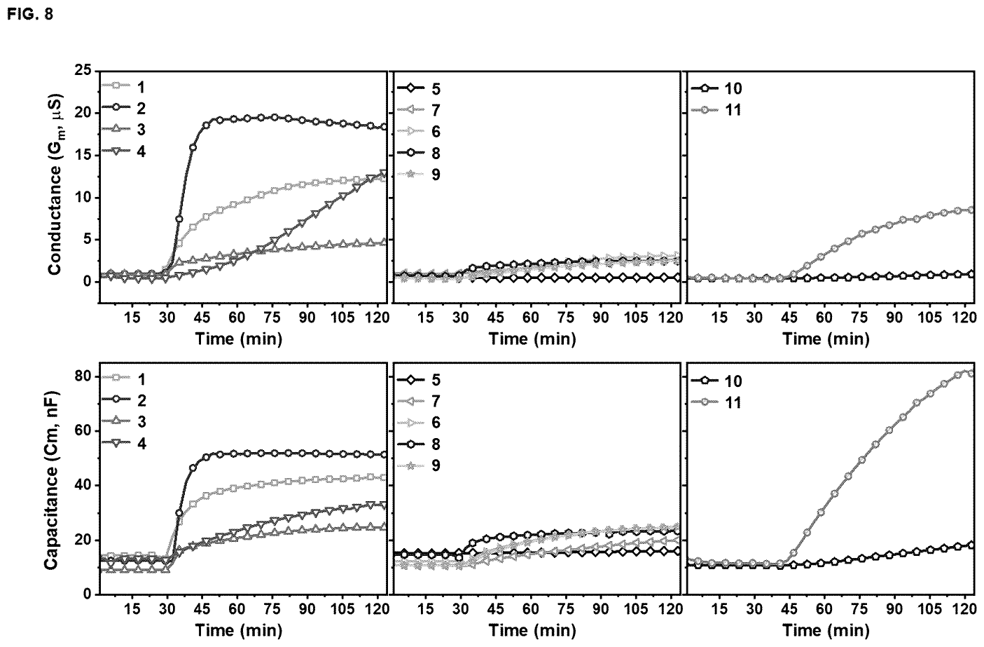

Importantly, the QCM-D experiments showed that all peptides, at 13 μM bulk peptide concentrations, could rupture surface-adsorbed lipid vesicles (FIG. 4). Additional QCM-D experiments showed that the peptides can interact with supported lipid bilayers in distinct ways depending on the mechanism of action (FIG. 5). Using time-lapse fluorescence microscopy imaging, highly parallel measurements on tethered lipid vesicles demonstrating that 10-1000 nM peptide concentrations induced vesicle rupture—at varying speeds and to varying extents—in a concentration- and sequence-dependent manner (FIG. 6). Detailed analysis revealed differences that certain peptides formed pores and induced vesicle rupture more quickly than other peptides (FIG. 7 and Table 2). EIS experiments that detected membrane defect formation when 8 μM peptide was added to tethered lipid bilayers, provided additional evidence that the different peptides had distinct mechanisms of action (FIG. 8).

TABLE 2
Vesicle rupture times (min) for 100 and 1000 nM peptide
treatment based on rhodamine (Rh) and calcein (Cal) dye
release using time-lapse fluorescence microscopy imaging.

| | 100 nM | | 1000 nM | |
|---|---|---|---|---|
| Peptide | Rh | Cal | Rh | Cal |
| 1 | 10.58 ± 0.33 | 5.95 ± 0.03 | 6.13 ± 0.09 | 4.67 ± 0.32 |
| 2 | 8.69 ± 0.08 | 5.54 ± 0.30 | 3.13 ± 0.12 | 1.91 ± 0.04 |
| 3 | 8.41 ± 0.08 | 4.53 ± 0.17 | 2.99 ± 0.03 | 1.97 ± 0.11 |
| 4 | 21.13 ± 0.38 | 6.50 ± 0.3 | 9.51 ± 0.09 | 3.71 ± 0.07 |
| 5 | 15.25 ± 0.43 | 4.69 ± 0.22 | 11.20 ± 0.17 | 2.53 ± 0.10 |
| 6 | 4.75 ± 0.06 | 2.46 ± 0.02 | 2.74 ± 0.03 | 1.57 ± 0.03 |
| 7 | 5.50 ± 0.11 | 2.41 ± 0.06 | 2.13 ± 0.00 | 1.47 ± 0.01 |
| 8 | 3.28 ± 0.08 | 2.49 ± 0.06 | 1.78 ± 0.02 | 1.70 ± 0.02 |
| 9 | 6.50 ± 0.07 | 4.22 ± 0.17 | 1.87 ± 0.014 | 1.41 ± 0.03 |
| 10 | 10.09 ± 0.10 | 2.38 ± 0.05 | 2.09 ± 0.05 | 2.06 ± 0.02 |
| 11 | 6.00 ± 0.13 | 4.15 ± 0.04 | 4.91 ± 0.38 | 4.67 ± 0.08 |

We further tested the in vitro antiviral activity of the peptides using a plaque reduction neutralization assay and all the peptides had high levels of inhibitory activity against all four Dengue serotypes (Table 3). Some of the peptides had significantly greater activity than that of Peptide 2, which had been tested before.

TABLE 3
50% plaque reduction neutralization titer
(PRNT₅₀) values determined for peptides with different
terminal caps against Dengue virus serotypes 1-4.

| | DENV-1 | DENV-2 | DENV-3 | DENV-4 |
|---|---|---|---|---|
| | PRNT₅₀ | PRNT₅₀ | PRNT₅₀ | PRNT₅₀ |
| Peptide | (μM) | (μM) | (μM) | (μM) |
| N1O | 0.2188 | 0.3626 | 0.9738 | 0.2935 |
| N2O | 0.1460 | 0.3841 | 0.6617 | 0.2663 |
| N3O | 0.1823 | 0.3078 | 1.019 | 0.2885 |
| N4O | 0.2781 | 0.5562 | 2.792 | 0.3332 |
| N5O | 0.2352 | 0.8385 | 1.591 | 0.6489 |
| N6O | 0.2182 | 0.5540 | 1.473 | 0.2733 |
| N7O | 0.7231 | 1.884 | 2.376 | 0.1977 |
| N8O | 0.2242 | 0.2573 | 0.6485 | 0.3433 |
| N9O | 0.1389 | 0.1853 | 0.2625 | 0.2395 |
| N100 | 0.1940 | 0.3615 | 1.110 | 1.149 |
| N11O | 0.1309 | 0.2802 | 0.9466 | 0.2755 |
| N1N | 0.2175 | 0.4015 | 3.226 | 1.104 |
| N2N | 0.1147 | 0.2897 | 0.4365 | 0.1712 |
| N3N | 0.1206 | 0.3891 | 0.9361 | 0.1390 |
| N4N | 0.2811 | 1.349 | 3.879 | 0.9840 |
| N5N | 0.2365 | 0.5450 | 1.381 | 0.3382 |
| N6N | 0.1624 | 0.5365 | 2.779 | 0.1652 |
| N7N | 0.1713 | 0.5620 | 1.142 | 0.2419 |
| N8N | 0.0974 | 0.2532 | 0.2710 | 0.1940 |
| N9N | 0.0969 | 0.5499 | 0.4765 | 0.0802 |
| N10N | 0.2012 | 0.9361 | 0.2587 | 0.7201 |
| N11N | 0.5909 | 0.924 | 0.2892 | 0.3060 |
| O1O | 0.1319 | 0.2419 | 0.1750 | 0.2865 |
| O3O | 0.1479 | 0.2919 | 0.2374 | 0.2668 |
| O4O | 0.3654 | 1.212 | 2.186 | 1.559 |
| O5O | 0.0372 | 0.1636 | 0.1763 | 0.2785 |
| O6O | 0.0680 | 0.4124 | 0.3335 | 0.2729 |
| O7O | 0.0360 | 0.2986 | 0.2128 | 0.4333 |
| O8O | 0.0692 | 0.1875 | 0.4438 | 0.7086 |
| O9O | 0.0500 | 0.1925 | 0.1187 | 0.1389 |
| O100 | 0.0603 | 0.3955 | 0.3160 | 0.5673 |
| O110 | 0.2579 | 1.160 | 0.4905 | 0.8102 |

The biophysical evidence show that the AH peptides can form pores in highly curved membranes of virion dimensions; in contrast, the AH peptide has negligible effect on much larger, and hence lower curvature, mammalian cell membranes. This discrimination supports a high degree of selectivity for targeting a common membrane structural element shared by all mosquito-borne enveloped viruses, including dengue, Zika, yellow fever, West Nile, and chikungunya viruses.

### Example 4. Peptides with Engineered Terminal Endings

The peptides prepared in Example 1 were engineered to have either amine (N) or carboxylic acid (O) functional groups. CD spectroscopy was performed by following the protocol in Example 2 while QCM-D and time-lapse fluorescence microscopy imaging studies were performed by following the protocol in Example 3.

Synthesis of Peptides with N or O Terminal Endings

N-terminal acetylation or C-terminal amidation was achieved by adding an acetyl or amide group to the appropriate terminal as part of the solid-phase peptide synthesis (G. Marino, U. Eckhard & C. M. Overall, ACS Chem. Biol. 2015, 10, 1754-1764).

### Results and Discussion

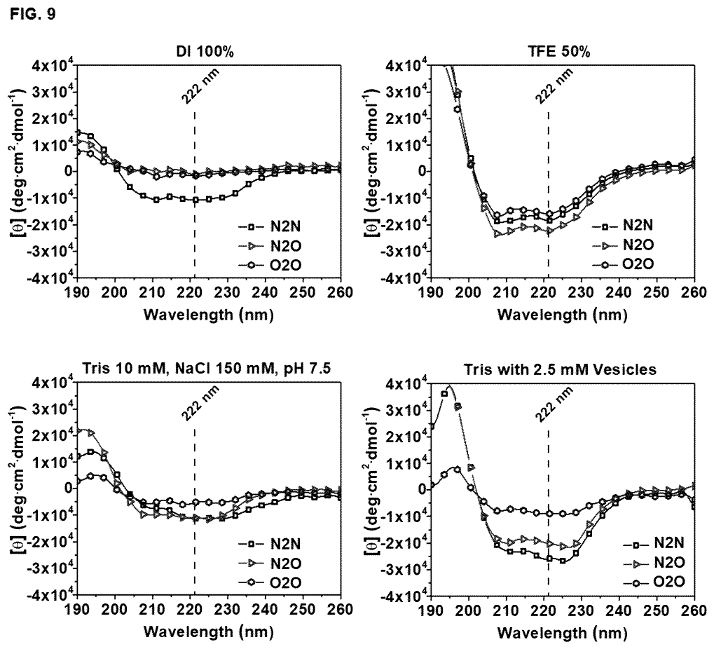

Using CD spectroscopy, we further explored Peptide 2 and saw that engineering the terminal endings to have either N or O functional groups could change the secondary structure in water and also affect the degree of coil-to-helix transition in hydrophobic environments, such as 50% TFE and lipid vesicle partitioning (FIG. 9). QCM-D experiments further showed that changing the terminal endings of Peptides 2 and 4 had a dramatic effect on the speed and degree of vesicle rupture and hence this modification strategy provides unexpected and remarkable control over membrane-peptide interactions based on conformational tuning (FIG. 10).

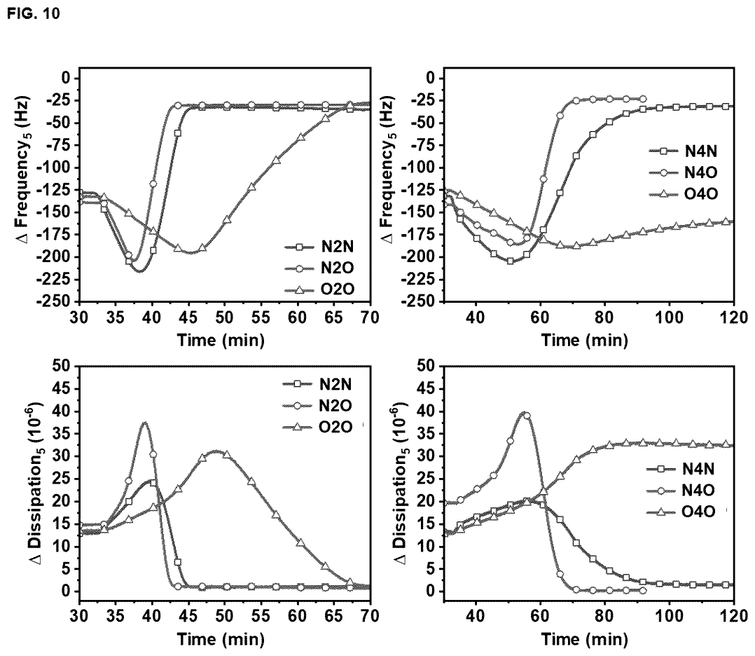

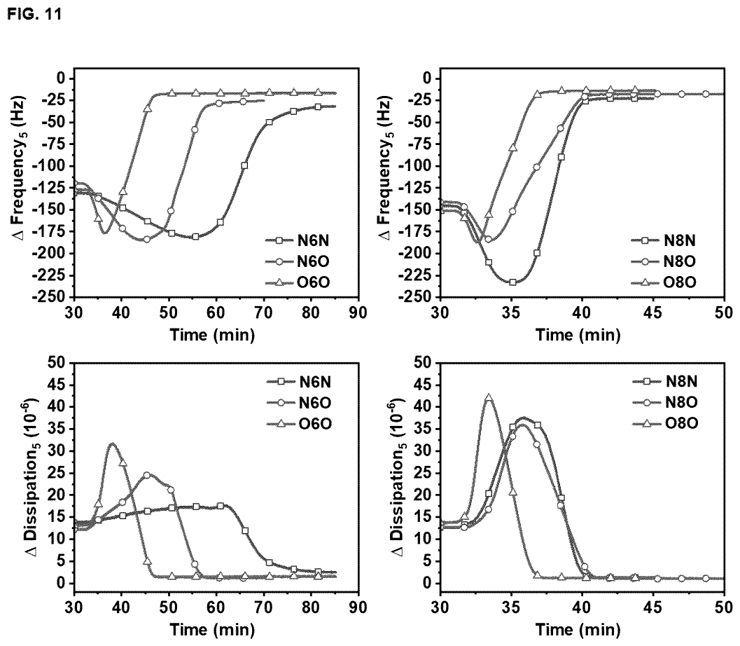

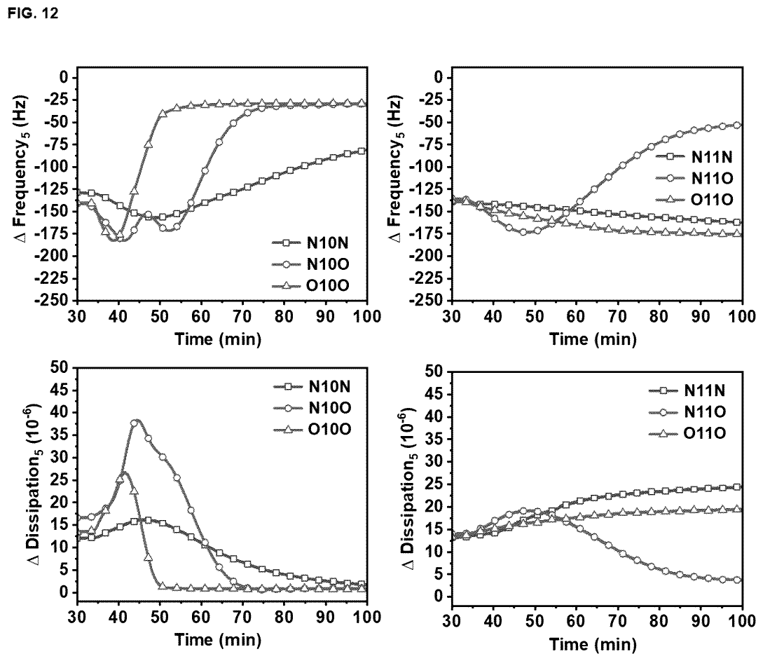

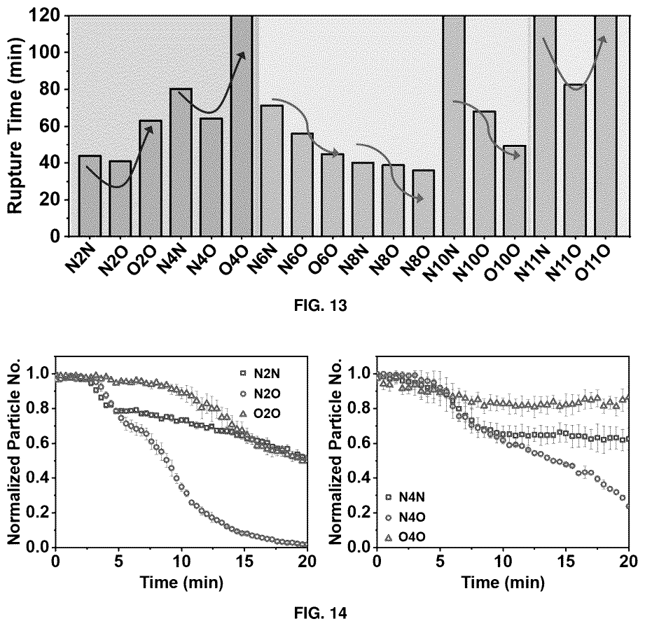

Using the QCM-D technique, similar levels of control were also demonstrated with Peptides 6, 8, 10, and 11 (FIG. 11-12). The specific degree of control over membrane-disruptive activity is dependent on the amino acid sequence and hence can be rationally controlled based on the structure-function relationship and conformational tuning (FIG. 13).

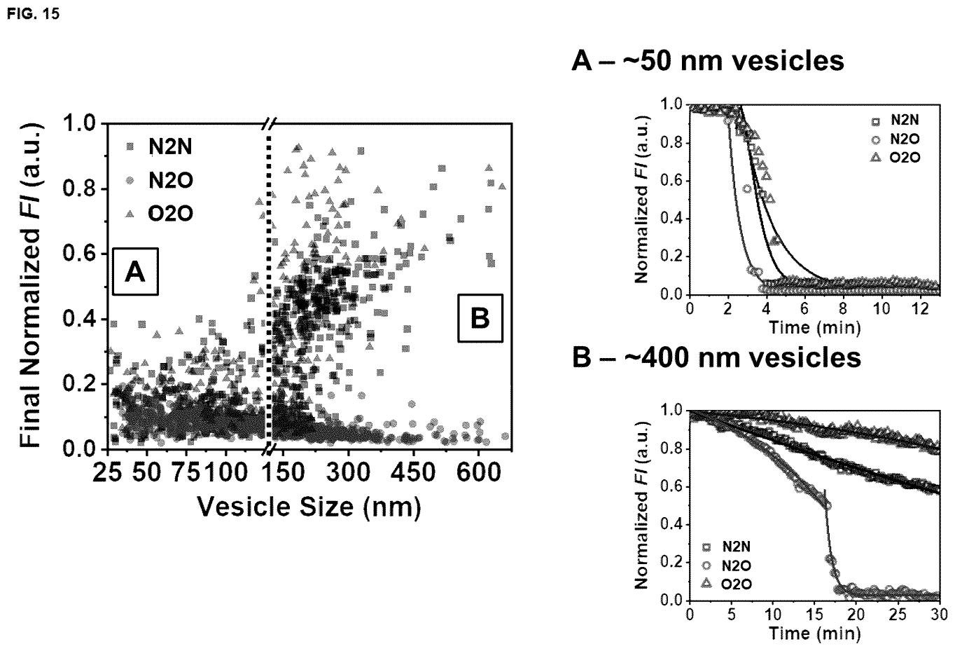

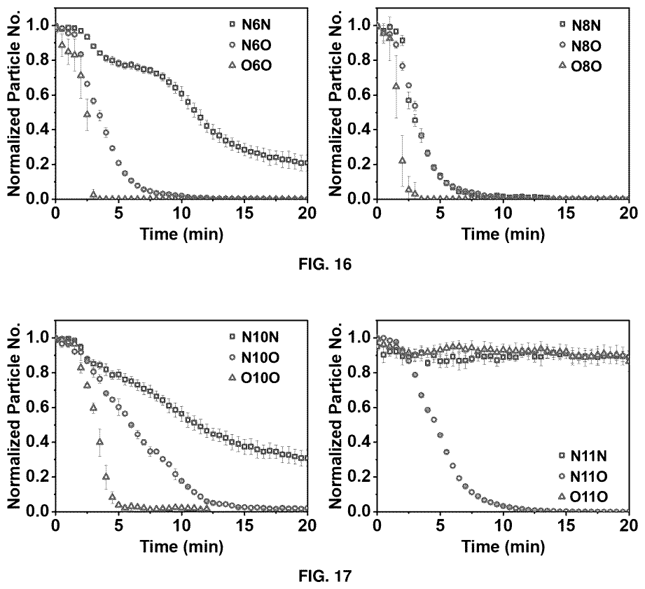

Using time-lapse fluorescence microscopy imaging, we further investigated the interactions of different versions of Peptides 2 and 4 with tethered vesicles at the single-vesicle level and saw different degrees of vesicle rupture in terms of total extent and speed (FIG. 14). Importantly, single-vesicle analysis showed that Peptides N2N and O2O have membrane-curvature sensing mechanisms while Peptide N2O ruptured vesicles independently of vesicle size—this is a key example of the importance of sequence tuning and installing fine modifications to drive critical behaviors that are related to potential therapeutic applications (FIG. 15). Using time-lapse fluorescence microscopy, similar levels of control were also demonstrated with Peptides 6, 8, 10, and 11 (FIG. 16-17).

### Example 5. Peptides from D-Amino Acids

In order to improve the pharmacological properties of peptide 2, the conventional L-amino acids were replaced by D-amino acids that are more resistant to proteolytic degradation, to give AH-D peptide.

### Blood-to-Brain Influx Assessment

Female, Institute for Cancer Research, Caesarean Derived-1 (ICR-CD-1) mice (7 to 10 weeks old; Envigo) were used to assess the in vivo blood-brain barrier (BBB) transport properties of AH-D peptide. The BBB permeability of AH-D was evaluated according to multiple time regression (MTR) analysis. In brief, mice were anesthetized by injecting 40% urethane intraperitoneally. Approximately 200 μl of AH-D peptide solution (10 mg kg⁻¹) was injected into the jugular internalis vein, then blood samples were collected from the carotid artery at selected time points after injection (minimum five time points per compound). Immediately after blood collection, the mice were euthanized by decapitation. The collected blood samples were centrifuged at 10,000 g for 15 min at 21° C. and the brains were isolated. The brain and the serum of each mouse were then analysed using a validated bio-analytical, ultrahigh-performance liquid chromatography tandem mass spectrometry method. Modelling of the data was performed according to the linear Gjedde-Patlak equation, giving the influx rate constant Kᵢₙ (unidirectional clearance) of AH-D peptide. The molar concentration in the brain was also calculated by converting the mass of peptide per mass of brain tissue into the mass of peptide per volume of brain tissue by assuming the brain density to be 1.04 g ml⁻¹. As negative and positive controls, ¹²⁵I-labelled BSA protein and dermorphin peptide were tested, respectively.

### Pharmacokinetic Characterization

Male BALB/c mice (InVivos) aged 8-9 weeks were used during the pharmacokinetic characterization experiments. In brief, mice were anaesthetized by isoflurane. AH-D peptide solution (25 mg kg⁻¹) was then administered by the intravenous (i.v.) route (tail vein). Blood samples were taken by cardiac puncture under isoflurane anaesthesia at regular time points after injection (eight time points per compound). The administered dose was maintained at 5 ml kg⁻¹. Selected organs were collected after euthanasia and were stored at −80° C. until bioanalysis. Blood samples were mixed with deionized water while tissue samples were homogenized in deionized water before extraction. The samples were then treated with phosphoric acid (5 μl), followed by acetonitrile (25 μl) that contained 15 ng ml⁻¹ imipramine internal standard, 5% ammonium hydroxide (25 μl), deionized water (100 μl) and 5% Tween-20 (20 μl), before vortexing. The processed samples were then added to pre-activated Strata-X Solid Phase Extraction cartridges (30 mg per 1 ml; Phenomenex), and washed with 500 μl of deionized water, followed by elution in 75/25 acetonitrile/methanol with 1% formic acid. The elute was air-dried under nitrogen gas and reconstituted in a 150 μl volume of 1:1 acetonitrile/water before sample injection. The blood and tissue samples were then analysed using a validated bio-analytical, ultra-performance liquid chromatography tandem mass spectrometry method. Data acquisition was done by MassLynx 4.1 mass spectrometry software and processed by TargetLynx Application Manager software (Waters Corporation). Pharmacokinetic analysis was performed using Phoenix WinNonlin software (Pharsight).

### Results and Discussion

This design choice enabled an improved pharmacokinetic profile, with a relatively long circulation half-time, high bioavailability, and BBB-crossing activity to achieve therapeutic concentrations in the brain. Surprisingly, even though the lipid membrane is an achiral target, the D-amino acid version of peptide 2 exhibited greater rupture potency and distinct membrane-curvature-sensing activity against model liposomes, as compared to the L-amino acid version.

### Example 6. Inhibition of Viral Infection in the Brain

### Inhibition of Viral Infection in Brain

SV129 IFN-α/βR^{-/-} (A129^{-/-}) mice were anaesthetized with 5% isoflurane before intracranial inoculation with 2×10⁵ p.f.u. ZIKV HS-2015-BA-01 in a total volume of 20 μl. Mice were euthanized on day 3 post-infection and viral loads in collected brain samples were quantified by plaque assay. AH-D peptide (5% DMSO) was administered twice daily (every 12 h) by the i.p. route at a dose of 25 mg kg⁻¹, and treatment was started either 1 day or 1 h before infection or 1 day after infection. Another control group was infected with ZIKV, and then treated with PBS (5% DMSO) instead of AH-D peptide.

### Results and Discussion

In addition to controlling systemic infection, the engineered AH-D peptide is able to cross the blood brain barrier (BBB) to reduce viral loads in the brain and protect against Zika virus-induced brain injury. The ability to inhibit viral infection in the brain is particularly important because the BBB can remain intact after neuroinvasion, and other classes of therapeutic molecules such as antibodies typically cannot cross the intact BBB. These findings indicate that the AH-D peptide can potentially be used to treat neurotropic infections through both systemic and neurological routes. The AH-D peptide should be tested against other mosquito-borne enveloped viruses in animal models. Taken together, the aforementioned progress highlights the molecular engineering of LEAD drug candidates as an exciting frontier at the convergence of infectious diseases, biochemistry, biophysics, and engineering.

1. A peptide of 27 amino acid residues in length comprising the following amino acid sequence:
X₁X₂X₃WLX₄X₅X₆WX₇WX₈X₉X₁₀X₁₁X₁₂X₁₃DFX₁₄X₁₅X₁₆LX₁₇X₁₈KX₁₉, wherein:
X₁ is S, Q D, or A;
X₂ is Q D, E, or S;
X₃ is S, D, N, or T;
X₄ is R, H, Y, or W;
X₅ is D, I, T, or E;
X₆ is I or V;
X₇ is D, E, or N;
X₈ is I or V;
X₉ is C or L;
X₁₀ is E, T, S, I, or H;
X₁₁ is I or V;
X₁₂ is L or V;
X₁₃ is S, T, or A;
X₁₄ is K or R;
X₁₅ is T, N, A, V or L;
X₁₆ is W or C;
X₁₇ is K, Q, T, S, or G;
X₁₈ is A or S; and
X₁₉ is L, I, or F (SEQ ID NO: 12)
and wherein:
each amino acid is independently an L- or D-amino acid;
the functional groups of the N- and C-termini of the peptide are:
(A) unchanged from the standard N- and C-termini functional groups;
(B) reversed, such that the N-terminus has a carboxylic acid functional group and the C-terminus has an amino functional group;
(C) the N-terminus and C-terminus both have an amino functional group; or
(D) the N-terminus and C-terminus both have a carboxylic acid functional group,
provided that, when the peptide has the sequence:
SGSWLRDVWDWICTVLTDFKTWLQSKL (SEQ ID NO: 2), the functional groups of the N- and C-termini of the peptide are selected from (B) to (D) above only.

2. The peptide according to claim 1, wherein the peptide comprises the following amino acid sequence:
X₁X₂X₃WLRX₅X₆WDWX₈CX₁₀X₁₁X₁₂X₁₃DFKX₁₅WLX₁₇X₁₈KX₁₉
wherein:
X₁ is S or G;
X₂ is G or D;
X₃ is S, D or T;
X₅ is D or I;
X₆ is I or V;
X₈ is I or V;
X₁₀ is T or S;
X₁₁ is I or V;
X₁₂ is L or V;
X₁₃ is S, Tor A;
X₁₅ is T, N or A;
X₁₇ is K or S;
X₁₈ is A or S; and
X₁₉ is L or I (SEQ ID NO: 13)
and wherein each amino acid is independently an L- or D-amino acid.

3. The peptide according to claim 1, wherein the peptide comprises the following amino acid sequence:
SX₂X₃WLRDX₆WDWVCX₁₀VLX₁₃DFKX₁₅WLX₁₇X₁₈KX₁₉
wherein:
X₂ is G or D;
X₃ is S, D or T;
X₆ is I or V;
X₁₀ is T or S;
X₁₃ is S or T;
X₁₅ is T, N or A;
X₁₇ is K or S;
X₁₈ is A or S; and
X₁₉ is L or I (SEQ ID NO: 14)
and wherein each amino acid is independently an L- or D-amino acid.

4. The peptide according to claim 1, wherein the peptide comprises the following amino acid sequence:
SGX₃WLRDIWDWVCX₁₀VLX₁₃DFKTWLSAKX₁₉
wherein:
X₃ is S, D or T;
X₁₀ is T or S;
X₁₃ is S or T; and
X₁₉ is L or I (SEQ ID NO: 15)
and wherein each amino acid is independently an L- or D-amino acid.

5. The peptide according to claim 1, wherein the peptide has a peptide sequence from the following list:
(i)
(SEQ ID NO: 1)
SGSWLRDIWDWICEVLSDFKTWLKAKL;
(ii)
(SEQ ID NO: 2)
SGSWLRDVWDWICTVLTDFKTWLQSKL;
(iii)
(SEQ ID NO: 3)
SGSWLRDVWDWVCTILTDFKNWLTSKL;
(iv)
(SEQ ID NO: 4)
SGSWLRDIWEWVCSILTDFKNWLSAKL;
(v)
(SEQ ID NO: 5)
SDDWLRIIWDWVCSVVSDFKAWLSAKI;
(vi)
(SEQ ID NO: 6)
SGDWLRIIWDWVCSVVSDFKTWLSAKI;
(vii)
(SEQ ID NO: 7)
SDDWLRTIWDWVCSVLADFKAWLSAKI;
(viii)
(SEQ ID NO: 8)
GDDWLHDIWDWVCIVLSDFKTWLSAKI;
(ix)
(SEQ ID NO: 9)
DGNWLYDIWNWVCTVLADFKLWLGAKI;
(x)
(SEQ ID NO: 10)
AESWLWEVWDWVLHVLSDFKTCLKAKF;
and
(xi)
(SEQ ID NO: 11)
GSTWLRDIWDWVCTVLSDFRVWLKSKL,
and wherein each amino acid is independently an L- or D-amino acid.

6. The peptide according to claim 1, wherein each amino acid in each sequence is an L-amino acid.

7. The peptide according to claim 1, wherein each amino acid in each sequence is a D-amino acid.

8. The peptide according to claim 1, wherein the amino acids in each sequence are a mixture of L- and D-amino acids.

9. The peptide according to claim 1, wherein the functional groups of the N- and C-termini of the peptide are:
(AI) the N-terminus and C-terminus both have an amino functional group; or
(AII) the N-terminus and C-terminus both have a carboxylic acid functional group.

10. A pegylated peptide comprising a peptide of 27 amino acid residues in length as described in claim 1 linked to one, two or more polyethylene glycol (PEG) polymers, optionally wherein:
(aa) the one, two or more PEG polymers is linked to the N-terminus and/or C-terminus of the peptide; and/or
(ab) each of the one, two or more PEG polymers has a molecular weight range of from 500 to 5,000 Daltons;
(ac) each of the one, two or more PEG polymers is branched or unbranched.

11. A pharmaceutical composition comprising a peptide according to claim 1 or a pegylated peptide according to claim 10, and a pharmaceutically acceptable carrier.

12. The pharmaceutical composition according to claim 11, wherein the composition further comprises one or more pharmaceutically acceptable excipients and adjuvants.

13. The pharmaceutical composition according to claim 11, wherein the composition is formulated for subcutaneous, intravenous or intraperitoneal administration.

14. A vial containing a pharmaceutical composition according to claim 11, optionally wherein the pharmaceutical composition is lyophilized.

15. A peptide according to claim 1, or a pegylated peptide according to claim 10 for use in medicine.

16. A method of treating or preventing a viral infection in a subject, the method comprising administering a pharmaceutically effective amount of a peptide according to claim 1, or a pegylated peptide according to claim 10.

17. A peptide according to claim 1, or a pegylated peptide according to claim 10 for use in treating or preventing a viral infection.

18. (canceled)

19. The method according to claim 16, the peptide or pegylated peptide for use according to claim 17, wherein the viral infection is selected from one or more of the group consisting of a virus of the coronaviridae or, more particularly, flaviviridae, togaviridae, filoviridae, arenaviridae, poxviridae, bunyaviridae, and retroviridae families.

20. The method, peptide or pegylated peptide for use, or use according to claim 19, wherein the viral infection is a mosquito-borne virus, optionally wherein the viral infection is selected from one or more of the group consisting of dengue, Zika, yellow fever, West Nile, and Chikungunya.

21. The method, peptide or pegylated peptide for use, or use according to claim 19, wherein the viral infection is selected from one or more of the group consisting of HCV, HDV, JEV, COVID19, SARS, MERS-CoV, MERS, SARS-CoV-2, alphacoronavirus, betacoronavirus, gammacoronavirus or, more particularly, dengue, Chikungunya virus, Ebola, HIV, West Nile, Zika, yellow fever, and influenza.


SEQUENCE LISTING (15 sequences)

SEQ ID NO: 1 - protein, 27 aa, Artificial Sequence
  Synthetic_Anti-viral peptide derived from hepatitis C virus NS5A protein
SGSWLRDIWDWICEVLSDFKTWLKAKL

SEQ ID NO: 2 - protein, 27 aa, Artificial Sequence
  Synthetic_Anti-viral peptide derived from hepatitis C virus NS5A protein
SGSWLRDVWDWICTVLTDFKTWLQSKL

SEQ ID NO: 3 - protein, 27 aa, Artificial Sequence
  Synthetic_Anti-viral peptide derived from hepatitis C virus NS5A protein
SGSWLRDVWDWVCTILTDFKNWLTSKL

SEQ ID NO: 4 - protein, 27 aa, Artificial Sequence
  Synthetic_Anti-viral peptide derived from hepatitis C virus NS5A protein
SGSWLRDIWEWVCSILTDFKNWLSAKL

SEQ ID NO: 5 - protein, 27 aa, Artificial Sequence
  Synthetic_Anti-viral peptide derived from hepatitis C virus NS5A protein
SDDWLRIIWDWVCSVVSDFKAWLSAKI

SEQ ID NO: 6 - protein, 27 aa, Artificial Sequence
  Synthetic_Anti-viral peptide derived from hepatitis C virus NS5A protein
SGDWLRIIWDWVCSVVSDFKTWLSAKI

SEQ ID NO: 7 - protein, 27 aa, Artificial Sequence
  Synthetic_Anti-viral peptide derived from hepatitis C virus NS5A protein
SDDWLRTIWDWVCSVLADFKAWLSAKI

SEQ ID NO: 8 - protein, 27 aa, Artificial Sequence
  Synthetic_Anti-viral peptide derived from hepatitis C virus NS5A protein
GDDWLHDIWDWVCIVLSDFKTWLSAKI

SEQ ID NO: 9 - protein, 27 aa, Artificial Sequence
  Synthetic_Anti-viral peptide derived from hepatitis C virus NS5A protein
DGNWLYDIWNWVCTVLADFKLWLGAKI

SEQ ID NO: 10 - protein, 27 aa, Artificial Sequence
  Synthetic_Anti-viral peptide derived from hepatitis C virus NS5A protein
AESWLWEVWDWVLHVLSDFKTCLKAKF

SEQ ID NO: 11 - protein, 27 aa, Artificial Sequence
  Synthetic_Anti-viral peptide derived from hepatitis C virus NS5A protein
GSTWLRDIWDWVCTVLSDFRVWLKSKL

SEQ ID NO: 12 - protein, 27 aa, Artificial Sequence
  Synthetic_Anti-viral peptide derived from hepatitis C virus NS5A protein
  MOD_RES (1): Xaa is Ser, Gly, Asp or Ala
  MOD_RES (2): Xaa is Gly, Asp, Glu or Ser
  MOD_RES (3): Xaa is Ser, Asp, Asn or Thr
  MOD_RES (6): Xaa is Arg, His, Tyr or Trp
  MOD_RES (7): Xaa is Asp, Ile, Thr or Glu
  MOD_RES (8): Xaa is Ile or Val
  MOD_RES (10): Xaa is Asp, Glu or Asn
  MOD_RES (12): Xaa is Ile or Val
  MOD_RES (13): Xaa is Cys or Leu
  MOD_RES (14): Xaa is Glu, Thr, Ser, Ile or His
  MOD_RES (15): Xaa is Ile or Val
  MOD_RES (16): Xaa is Leu or Val
  MOD_RES (17): Xaa is Ser, Thr or Ala
  MOD_RES (20): Xaa is Lys or Arg
  MOD_RES (21): Xaa is Thr, Asn, Ala, Val or Leu
  MOD_RES (22): Xaa is Trp or Cys
  MOD_RES (24): Xaa is Lys, Gln, Thr, Ser or Gly
  MOD_RES (25): Xaa is Ala or Ser
  MOD_RES (27): Xaa is Leu, Ile or Phe
XXXWLXXXWXWXXXXXXDFXXXLXXKX

SEQ ID NO: 13 - protein, 27 aa, Artificial Sequence
  Synthetic_Anti-viral peptide derived from hepatitis C virus NS5A protein
  MOD_RES (1): Xaa is Ser, or Gly
  MOD_RES (2): Xaa is Gly, or Asp
  MOD_RES (3): Xaa is Ser, Asp or Thr
  MOD_RES (7): Xaa is Asp, or Ile
  MOD_RES (8): Xaa is Ile or Val
  MOD_RES (12): Xaa is Ile or Val
  MOD_RES (14): Xaa is Thr or Ser
  MOD_RES (15): Xaa is Ile or Val
  MOD_RES (16): Xaa is Leu or Val
  MOD_RES (17): Xaa is Ser, Thr or Ala
  MOD_RES (21): Xaa is Thr, Asn, or Ala
  MOD_RES (24): Xaa is Lys or Ser
  MOD_RES (25): Xaa is Ala or Ser
  MOD_RES (27): Xaa is Leu or Ile
XXXWLRXXWDWXCXXXXDFKXWLXXKX

SEQ ID NO: 14 - protein, 27 aa, Artificial Sequence
  Synthetic_Anti-viral peptide derived from hepatitis C virus NS5A protein
  MOD_RES (2): Xaa is Gly or Asp
  MOD_RES (3): Xaa is Ser, Asp or Thr
  MOD_RES (8): Xaa is Ile or Val
  MOD_RES (14): Xaa is Thr or Ser
  MOD_RES (17): Xaa is Se or, Thr
  MOD_RES (21): Xaa is Thr, Asn or Ala
  MOD_RES (24): Xaa is Lys or Ser
  MOD_RES (25): Xaa is Ala or Ser
  MOD_RES (27): Xaa is Leu or Ile
SXXWLRDXWDWVCXVLXDFKXWLXXKX

SEQ ID NO: 15 - protein, 27 aa, Artificial Sequence
  Synthetic_Anti-viral peptide derived from hepatitis C virus NS5A protein
  MOD_RES (3): Xaa is Ser, Asp or Thr
  MOD_RES (14): Xaa is Thr or Ser
  MOD_RES (17): Xaa is Ser or Thr
  MOD_RES (27): Xaa is Leu or Ile
SGXWLRDIWDWVCXVLXDFKTWLSAKX